(19) United States
(12) Patent Application Publication
(10) Pub. No.: US 2026/0209689 A1
(43) Pub. Date: Jul. 23, 2026

(54) MICROCAPSULE CROSSLINKING

(71) Applicant: Droplet Genomics, UAB, Vilnius (LT)

(72) Inventors: Giedrius ZOLUBAS, Vilnius (LT); Rapolas ZILIONIS, Vilnius (LT); Karolis LEONAVICIUS, Vilnius (LT); Greta LEONAVICIENE, Vilnius (LT)

(73) Assignee: Droplet Genomics, UAB, Vilnius (LT)

(21) Appl. No.: 19/396,726

(22) Filed: Nov. 21, 2025

Related U.S. Application Data
(63) Continuation of application No. PCT/EP2024/064312, filed on May 23, 2024.
(60) Provisional application No. 63/504,267, filed on May 25, 2023.

(51) Int. Cl. C12N 5/00; C12N 5/09

(52) U.S. Cl. CPC C12N 5/0012; C12N 5/0694; C12N 2537/10

(57) ABSTRACT

Approaches for the gentle formation of microcapsule populations are disclosed. Encapsulation is effected while reducing or eliminating negative impacts of hydrogel crosslinking in microcapsule contents, and using hydrogel reagents that may allow release of contents under physiological conditions. Also disclosed are the resultant microcapsule populations, which harbor contents such as various cell populations at proportions that reflect their relative proportions prior to encapsulation, such that encapsulation does not differentially select for one or another cell population.

### CROSS-REFERENCE TO RELATED APPLICATIONS

This document claims priority to U.S. Prov Ser. No. 63/504,267, filed May 25, 2023, the contents of which are hereby incorporated by reference in their entirety.

### BACKGROUND

Biocompatible hydrogels afford opportunities to contain macromolecules and even viable cells in solid compartments that are accessible to reagent exchange. However, formation of hydrogels often comprises contacting the macromolecules or viable cells to harsh biochemical conditions such as oxidative stress that may impact macromolecule activity or cell viability.

### SUMMARY

Disclosed herein are methods of crosslinking a capsule shell without exposing capsule contents in an interior space of the capsule to a chemical stress such as oxidative stress, at a level sufficient to impact or to substantially impact capsule contents integrity such as cell viability, transcriptome or protein accumulation levels, protein integrity, nucleic acid integrity, or other measure of content integrity. Similarly, some methods disclosed herein relate to crosslinking a capsule shell without exposing capsule contents in an interior space of the capsule to a chemical stress such as oxidative stress at a level sufficient to substantially impact cell contents viability, chemical activity, transcription accumulation levels, metabolism, or other metric of chemically induced perturbation.

Some such methods comprise one or more of preventing a chemical stress moiety from being active in the interior space of the capsule, and directing crosslinking to the periphery of the capsule rather than the interior. A partial list of chemical stress moieties includes oxidative stress moiety, a free radical moiety, a phosphoryl radical, benzoyl radical (such as an LAP [Lithium phenyl (2,4,6-trimethylbenzoyl) phosphinate cleavage radical), methyl radical as may be found on the shell polymer, a hydroxyl radical (OH·), superoxide anion (·O2-), hydrogen peroxide (H2O2), singlet oxygen (1O2), or ozone (O3), among others. The chemical stress moiety such as an oxidate stress moiety or free radical moiety is prevented from being active in the interior space by one or more of the following, exclusion or displacement of a chemical stress such as oxidative stress or free radical precursor from the space, or excluding or displacing a chemical stress such as oxidative stress or free radical induction moiety from the space, or inducing hydrogel formation without involving formation of a chemical stress moiety such as oxidative stress or free radical.

Similarly disclosed herein are systems and compositions used in or allowing the practice of the methods mentioned above.

Also disclosed herein are microcapsule compositions, (herein interchangeably referred to as SPC compositions) harboring biomolecules that are sensitive to free radical stress such as that used in hydrogel formation, but that are nonetheless not substantially impacted by the hydrogel formation process. In various cases, microcapsule populations comprise viable cells such as eukaryotic cells or even human cells at a frequency indicative of modest, minimal, or no substantial impact of the hydrogel microcapsule encapsulation process on cell viability, transcription activity, stress response, proliferative rate, or other measure of cell health. Microcapsule populations in some cases harbor contents that qualitatively, quantitatively, or both quantitatively and qualitatively reflect the distribution of the contents in a pre-encapsulated contents composition. In some cases microcapsule populations harbor viable cells at a frequency of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% of the frequency of viable cells in the emulsion from which the microcapsules were generated. In some cases cells exhibit a survival rate of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% when subjected to microcapsule formation. In some cases biomolecules exhibit a bioactivity survival rate of of at least 990, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% when subjected to microcapsule formation. In some cases nucleic acid molecules exhibit an effective concentration of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% of the concentration in the emulsion from which the microcapsules were generated. In some cases biomolecules exhibit an effective concentration of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% of the concentration in the emulsion from which the microcapsules were generated. In some cases biomolecules exhibit an effective activity of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% of the activity in the emulsion from which the microcapsules were generated.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 depicts an approach for microcapsule internal buffer replacement.

At FIG. 2, one sees Bright-field microscopy images showing K562 eukaryotic cell growth in microcapsules or SPCs generated by DTT-induced gelation followed by photopolymerization in aqueous suspension.

At FIG. 3, one sees Bright-field microscopy image showing K562 cells in SPCs generated by DTT-induced gelation followed by photopolymerization in aqueous suspension after six days in culture.

At FIG. 4, one sees a Bright Field microscopy image showing failure to form microcapsules able to survive 6 days under K562 growth conditions.

At FIG. 5 and FIG. 6, one sees Bright Field microscopy images showing stably formed microcapsules, some of which harbored proliferating K5623 cells at 6 days under K562 growth conditions.

At FIG. 7, one sees Bright Field microscopy images showing stably formed microcapsules in oil, generated using BAPO in carrier solution augmented with EHDAB.

At FIG. 8, one sees Bright Field microscopy images showing stably formed microcapsules resuspended in aqueous solution, generated using BAPO in carrier solution.

At FIG. 9, one sees Bright Field microscopy images showing stably formed microcapsules resuspended in aqueous solution, generated using BAPO in carrier solution augmented with EHDAB.

At FIG. 10 (split over two drawing pages) one sees a time course of cell growth post encapsulation in the absence, left, or presence, of Argon flushing during encapsulation.

At FIG. 11, one sees a time course of cell proliferation for two cell encapsulation approaches.

### DETAILED DESCRIPTION

### Introduction

Hydrogel microcapsules find increasing utility as versatile containers for iterative bioreactions. Hydrogel capsules comprise a shell, often a porous hydrogel shell, and an aqueous core comprising a local aqueous reaction environment. The local aqueous environment may also harbor contents derived from a sample and separated into sample subdivision that may be subjected to analysis such as nucleic acid sequencing or quantification, proteomic or individual protein analysis, culture reagent exposure, quantification or other analysis. Exemplary compositions to be subdivided into microcapsules include an aqueous sample lysate having biomolecules dissolved therein such as proteins or nucleic acids, or an aliquot of a sample comprising one or more discrete bioparticles, such as viable live cells, such as eukaryotic cells or human cells, biochemically active organelles, or protein or RNA complexes. Samples may be derived from sources comprising or suspected of comprising otherwise ‘unculturable’ cells, disarticulated or disassociated tissue samples such as disarticulated or disassociated tumors, cells in solution such as samples obtained from patient blood, or other samples comprising cells, viral particles, metabolites or other biomolecules of interest, among others Approaches herein are in some cases particularly suited for isolation and in some cases culturing of cells such as eukaryotic cells or human cells, rare cells, cells that exhibit differential or reduced viability in response to stress, or tissues having a diversity of cells, some of which differ as to viability or tolerance to encapsulation conditions such that encapsulation approaches that expose the diversity of cells to harsh chemical moieties such as oxidative stress may succeed in encapsulating a subset of the diversity of cells that is not representative of the full spectrum of the initial set.

A number of approaches have been proposed to produce capsules. In one such set of approaches, aqueous two-phase system (ATPS) droplets are generated using a microfluidics system and then the hydrogel shell phase layer is hardened by inducing polymerization. As exemplified in this approach, to achieve concentric ATPS droplet formation, a polymerizable hydrogel precursor and an aqueous, nonpolymerizable constituent are combined in the ATPS, and the hydrogel precursor is polymerized. In some examples, polymerizable density-matched Polyethylene Glycol Diacrylate (PEGDA) and Dextran polymer solutions can be used. Alternately, methacryloyl-modified dextran or acryloyl-modified dextran in one phase and a second phase of unmodified dextran are used to form an ATPS. A number of compositions are known to be compatible with microcapsule formation Some exemplary approaches use Dextran and DexMAB. A partial list of moieties for microcapsule formation includes click chemistry moieties, conjugated dienes, a dienophile, an azide, alkyne or nitrone moieties, thia-michael moieties such as maleimide, noncovalent binding moieties such as biotin-avidin or streptavidin combinations, ring-opening metathesis approaches such as those using cyclic olefins, or S—S bond formation such as is accomplished using thiol groups. Some exemplary microcapsule compositions comprise any polymer to which a crosslinking moiety may be added and which can be degraded under physiological or enzymatic conditions.

Microcapsules are formed in some cases by emulsifying the microcapsule precursors in an oil carrier, and then inducing the hydrogel precursor of the ATPS to crosslink. As the hydrogel precursor is likely to be drawn to the oil carrier at the droplet borders interfacing with the emulsion, it will accumulate at the edges of the droplets, such that upon crosslinking the hydrogel will from as a shell around the droplet's aqueous interior.

The process of microcapsule formation from ATPS droplets allows for the inclusion in the microcapsule of contents such as subdivisions of a sample, for example a sample comprising a plurality of viable cells or biomolecules. So long as the encapsulated entities are able to remain unchanged by the hydrogel induction process relatively to conditions where hydrogel formation is not induced, or even to survive the hydrogel induction process, the result is a microcapsule population where at least some of the microcapsules comprise sample subdivisions such as cells, particles or biomolecules.

Once a capsule is formed, the composition of its aqueous interior can be perturbed by adding new reagents or replacing old ones (e.g., by resuspending capsules in desired aqueous solution). The few limitations on these replacing reagents include that the hydrogel be permeable to them, and that they do not disrupt hydrogel integrity.

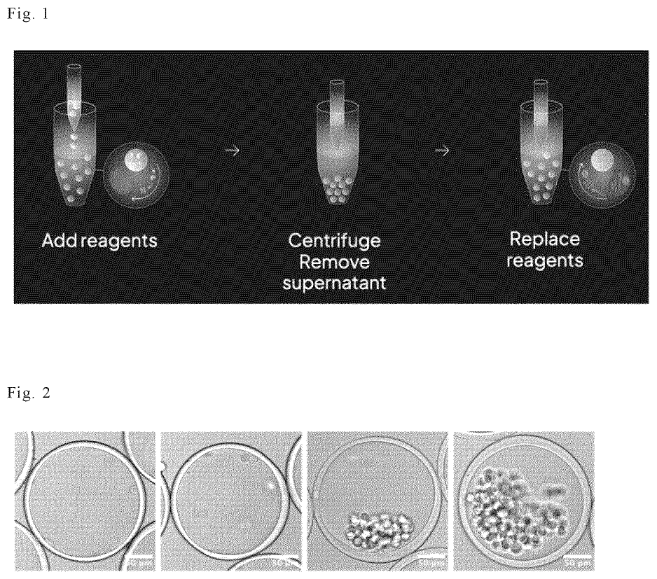

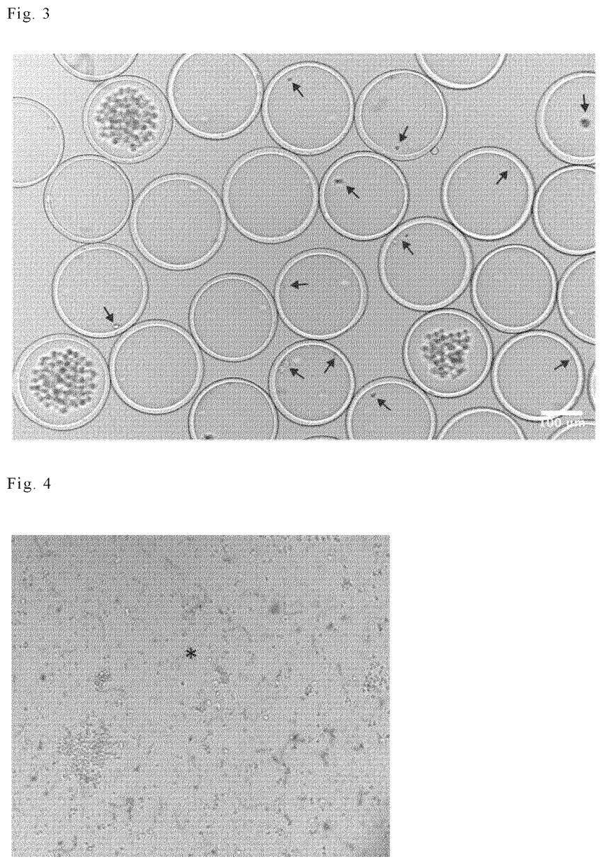

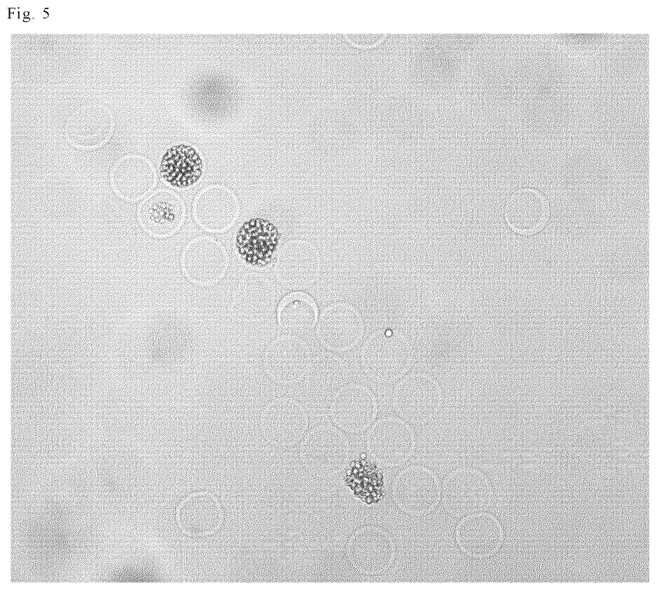

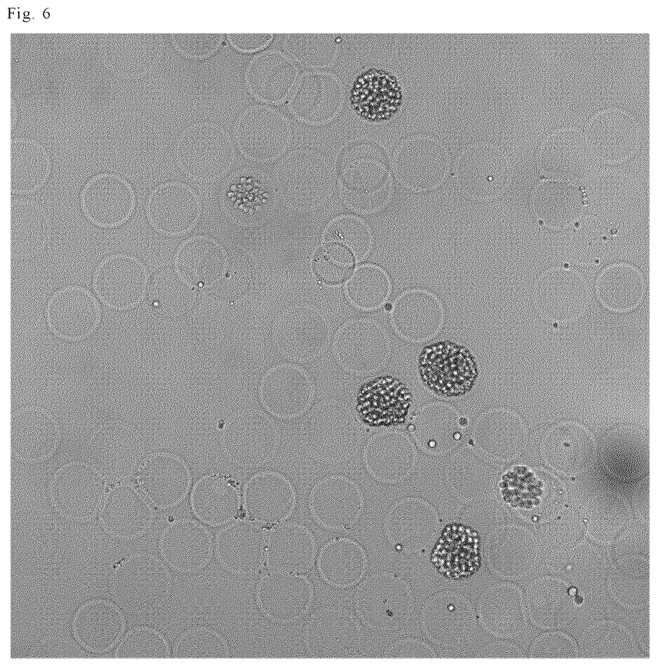

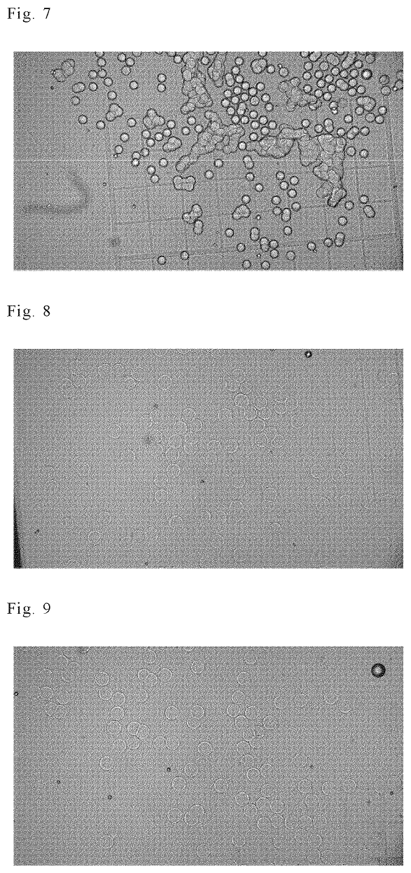

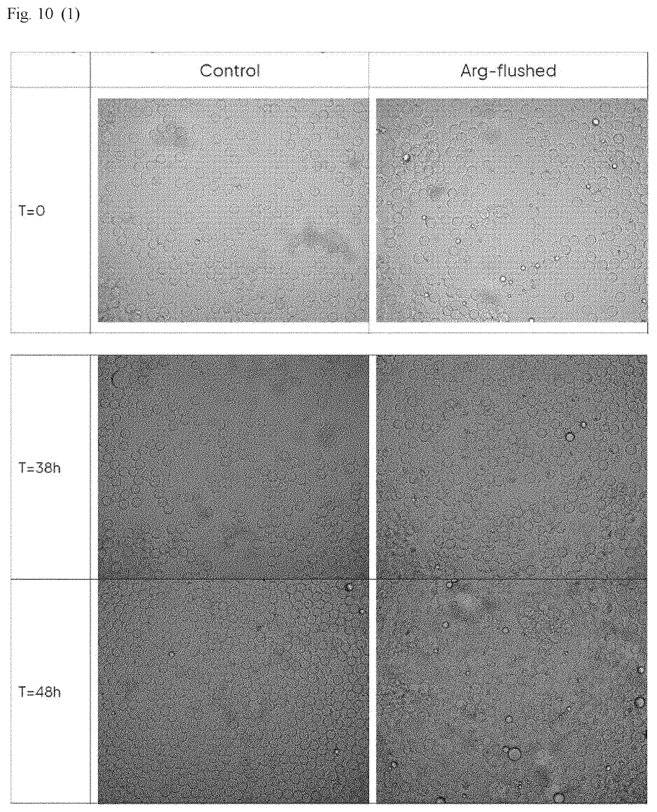

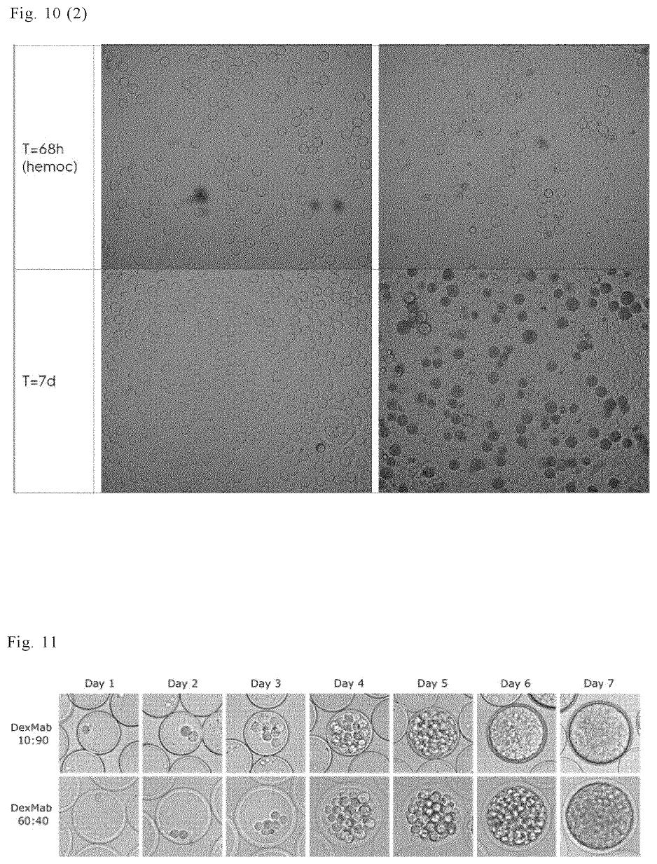

By regulating the hydrogel porosity, hydrogel microcapsules are synthesized that are porous to small molecule buffer constituents and to small enzymes and oligonucleotides, but retain large biomolecules, for example DNA of at least 500 bp, at least 600 bp, at least 700 bp, at least 800 bp, at least 900 bp, at least 1 kb, or other biomolecules of equivalent size. Alternately or in combination, hydrogel microcapsules are synthesized that are porous to small molecule buffer constituents and to small enzymes and oligonucleotides, but retain large biomolecules, for example being porous to nucleic acids of no greater than 50 bases, no greater than 100 bases, no greater than 200 bases, no greater than 300 bases, no greater than 400 bases, no greater than 500 bases, no greater than 600 bases, no greater than 700 bases, no greater than 800 bases, no greater than 900 bases, no greater than 1 kb, no greater than 1.5 kb, for example at least 500 bp, at least 600 bp, at least 700 bp, at least 800 bp, at least 900 bp, at least 1 kb, or other biomolecules of equivalent size. By incubating hydrogel microcapsules in a desired buffer environment, one allows the microcapsules to come to equilibrium with the buffer, effectively exchanging their previous internal reaction environment with that of their aqueous carrier, as shown in FIG. 1.

This approach is disclosed, for example, in US 2020/0400538, published Dec. 24, 2020, which is hereby incorporated by reference in its entirety.

Water in oil emulsions and well-borne reactions, in contrast, are largely limited to addition of reagent droplets or boluses to change their local aqueous reaction environment via dilution. These approaches are generally limited by the challenges of delivering reagent droplets of sufficient volume and concentration necessary to change the local aqueous reaction environment within the well or emulsion droplet without removal of pre-existing contents, which often has a substantial impact on total volume resulting from iterative droplet additions. In addition, the first reaction may include components, e.g., hydrolases, that even after a substantial dilution (100×, 1000×, or even more) interfere with the subsequent reaction.

Using porous hydrogel microcapsules, in contrast, one may readily adjust the local aqueous reaction environment within the microcapsule any number of times without impacting the microcapsule volume. An example of local aqueous reaction environment replacement is shown in FIG. 1.

One example of porous hydrogel microcapsule local aqueous reaction environment adjustment is that of growth medium supplementation. Through this approach, a viable cell or proliferating cell population, such as eukaryotic cells or human cells, contained within a microcapsule is provided with an ongoing optimal growth medium composition such that cell metabolism or proliferation is not hindered by exhaustion of components of the growth medium in the local aqueous reaction environment.

Similarly, a local aqueous reaction environment may be adjusted, for example through supplementation with a differentiation hormone or an expression regulator, so as to identify suitable growth conditions. For example, an environmental sample may be obtained and subdivided into microcapsules, which are then placed into a native growth environment medium, such as sea water, soil slurry or other complex growth environment. Encapsulated cells may be assayed for growth in native environments and in reconstituted growth media mimicking the native environment so as to identify factors needed for growth. Alternately, cells whose growth conditions are not known may nonetheless be sterilely grown in native environment conditions, such that they may be cultured and analyzed without needing to know the factors essential for growth. Expanded cells may then be isolated, released and subjected to downstream analysis. Accordingly, cells or cell populations may be readily maintained or manipulated in porous hydrogel microcapsules with a level of control not available in other sample subdivision systems such as water in oil emulsions or growth in microwells.

Containment of cells in hydrogels is well known, being disclosed in US 2020/0400538, published Dec. 24, 2020, which is hereby again incorporated by reference in its entirety, as well as US2021/0268465, published Sep. 2, 2021, and U.S. Pat. No. 8,765,485, published Jul. 1, 2014, both of which are hereby incorporated by reference in their respective entireties.

However, a challenge of approaches employed in the art as of the filing of the present application is that the process of hydrogel formation is often detrimental to cell viability, particularly for eukaryotic cells or human cells, such that a substantial proportion of microcapsules do not harbor viable cells, or a substantial proportion of viable cells in a sample do not survive the microcapsule encapsulation process, or do not survive the encapsulation process without being impacted at the level of growth rate, transcription profile, stress response expression or other response to the chemical or other stress of the encapsulation process. For example, the approach of Tamminen, M. V & Virta, M. P. J “Single gene-based distinction of individual microbial genomes from a mixed population of microbial cells.” Front. Microbiol. 6, 1-10 (2015) discloses that bacteria are encapsulated in acrylamide hydrogel beads, then the beads carrying embedded bacteria are re-suspended in warm agarose and emulsified again, in a process that leads to capsules with a hydrogel core composed of acrylamide and hydrogel-shell composed of agarose. The authors show that the hydrogel core can be dissolved using DTT, forming liquid core capsules contained within an agarose shell. Such a process succeeds in cell encapsulation, but is likely to impact viability as well as transcription profiles of expressed cells.

A further challenge in the analysis of encapsulated sample subdivisions is that, even if gentle microcapsule formation is accomplished, release of microcapsule contents requires harsh treatment. For example, relatively gentle radical free crosslinking of PEG-dextran capsules has been demonstrated, such as in van Zee et al (2022) “High-throughput selection of cells based on accumulated growth and division using PicoShell particles” PNAS 119(4) January 2022, or in Fattahi et al. (2021) “Core-shell hydrogel microcapsules enable formation of human pluripotent stem cell spheroids and their cultivation in a stirred bioreactor” Scientific Reports Vol 11, Article no. 7177. However, capsules may be formed without harm to the contents, but the contents cannot be released without exposing the contents to harsh degradants or other conditions that may impact the contents such as cells. Accordingly, the difficulty of releasing capsule contents has limited the efficacy of these methods.

### Gentle Cell Encapsulation and Release

The methods and compositions disclosed herein are substantially more conducive to cell viability, resulting in microcapsule compositions have high viable cell or other sample subdivision occupancy, or a viable cell occupancy that is reflective of cell or other sample component input concentration, with a low impact or level of stress exposure, encapsulated in microcapsules that are similarly degraded without substantial harm to microcapsule contents.

As a reference, a core approach to microcapsule formation is as follows. An ATPS comprising methacryloyl-modified dextran (DexMAB) or acryloyl-modified dextran in one phase and a second phase of unmodified dextran is emulsified in an oil carrier. The ATPS further comprises a photo-inducer of free radical formation such as lithium phenyl-2,4,6-trimethylbenzoylphosphinate (LAP). In some cases, the ATPS further comprises a sample that is subdivided into partitions prior to or pursuant to emulsification.

The emulsified microcapsule precursor droplets are subjected to photoinduction, such as by visible light, such as near ultraviolet light, or ultraviolet light, or other electromagnetic radiation so as to trigger oxygen free-radical accumulation in the droplet space. Some exemplary wavelengths are no less than, no greater than, about or exactly of 700 nm, 600 nm, 550 nm, 500 nm, 450 nm, 440 nm, 430 nm, 420 nm, 415 nm, 410 nm, 405 nm, 400 nm, 395 nm, 390 nm, 380 nm, 370 nm, 350 nm, 300n, or less than 300 nm. The free radicals trigger polymerization of the methacryloyl-modified dextran to form a hydrogel at the droplet perimeter, leaving the unmodified dextran in the aqueous core of the microcapsule, along with the sample subdivision.

This approach is very effective in producing microcapsules having consistent particle sizes and pore sizes, which allows for consistent, repeatable reagent replacement across microcapsules in a population, and further allow for gentle, enzymatic degradation of the hydrogel to release cell contents. The microcapsules are readily degraded under physiological conditions using a dextranase or other carbohydrate degrading enzyme such as a glycosidase, or using physiologically gentle chemical or thermal degradation. However, the process is in some cases harsh on the sample subdivisions to be contained in the microcapsules. In particular, the crosslinking process generates free radicals that may impact samples such that the encapsulated contents are either not viable or are substantially impacted by the encapsulation process that they no longer exhibit at least some of the properties that they possessed prior to encapsulation.

Accordingly, modifications to this core approach are provided. These modifications are presented in the context above, but are readily applicable to a broad range of microcapsule generation workflows, such as those for which an alternate ATPS or an alternate photo-inducer is used, or an alternate approach to hydrogel formation is taken. Commonalities of many of these alternatives is that they enable cell-friendly assembly of microcapsules, wherein the microcapsules may be degraded under similarly cell-friendly conditions. The term “cell-friendly” is understood to encompass conditions that facilitate cell survival or reduce impact on cells, and is more broadly to apply beyond cells because such conditions also prevent damage to macromolecules, organelles or protein or nucleic acid complexes by free-radicals.

Improvements to this method are presented below. Again, these improvements enable microcapsules that are formed, or both formed and degraded under cell-friendly conditions, such that sample subdivisions comprising viable cells can be encapsulated, manipulated and released.

Improvements apply generally to microcapsule formation with preservation of integrity of contents. These improvements generally relate to the protection of microcapsule aqueous core precursor contents from the harsh chemical moieties such as oxygen free radicals that induce hydrogel crosslinking but that may harm some sample subdivision contents, while effecting formation of microcapsules that are readily degraded under enzymatic or other cell-friendly conditions. In some embodiments the improvements are applied to the workflow above.

A number of approaches disclosed herein relate to protection of hydrogel microcapsule local aqueous reaction environment from potentially harsh effects of the microcapsule generation process. Consequently, contents of hydrogel microcapsule local aqueous reaction environments, such as cells, are more able to survive the process, resulting in microcapsule populations having high viable cell occupancy. Furthermore, the microcapsules in which they are contained are readily degraded under physiological conditions, such that the contents may be both encapsulated and recovered without harm.

Without being bound by theory, one potential source of sample perturbation such as harm to cell viability is chemical stress, such as oxidative stress, reactive oxygen species or free oxygen radicals, among others. Such stress is generated pursuant to hydrogel crosslinking induced by radical formation as is generated by LAP or other crosslinking inducers.

Sources of chemical stress are generally oxidized modifications of or radicals of small molecules that readily diffuse or are distributed throughout the aqueous phase of a microcapsule or microcapsule precursor, such that the contents of the microcapsule such as a cell or cells, or other bio-sensitive microcapsule payload, are exposed to and may potentially react with the source of chemical stress. Exemplary sources of chemical stress include Phosphoryl radicals, benzoyl radicals (such as LAP cleavage radicals), methyl radicals, such as may form on the shell polymer, hydroxyl radical (OH·), superoxide anion (·O2-), hydrogen peroxide (H2O2), singlet oxygen (1O2), and ozone (O3), among others. Chemical stress moieties act alone or in combination of, for example, 2, 3, 4, 5, or more than 5 of the listed chemical stresses, alone or in combination with unlisted chemical stresses contemplated herein or otherwise known. In many cases, the source of chemical stress is a hydroxy radical. Similarly, in many cases a source of chemical stress is hydrogen peroxide.

A chemical stress is then transferred to or exerted in the interior of the hydrogel microcapsule local aqueous reaction environment by one or more reactive molecule or chemical stress moieties such as free oxygen molecules in the hydrogel precursor emulsion carrier phase.

A number of crosslinking approaches may induce chemical stress such as oxidative stress or other stress arising from free radical formation such as oxygen free radical formation. In particular, some crosslinking approaches comprise an inducible, such as photoinducible, free-radical forming agent in the hydrogel precursor, in the local aqueous reaction environment precursor, or in both the hydrogel and aqueous environment precursor. Suitable photoinducers include electromagnetic radiation such as visible light, for example near ultraviolet visible light, or ultraviolet light. In some cases wavelengths suitable for crosslinking are no greater than 700 nm, 600 nm, 550 nm, 500 nm, 450 nm, 440 nm, 430 nm, 420 nm, 415 nm, 410 nm, 405 nm, 400 nm, 395 nm, 390 nm, 380 nm, 370 nm, 350 nm, 300n, or less than 300 nm.

These crosslinking approaches are effective at inducing crosslinked hydrogel formation from, for example, modified hexose or pentose hydrogel precursors. Exemplary precursors comprise methacryloyl-modified dextran, acryloyl-modified dextran, as well as a broad range of similarly modified hexose or pentose sugars, modified with any one of a broad range of moieties suitable for inducible crosslinking. By including unmodified hexose or pentose sugars such as unmodified dextran in an ATPS, one ensures that, concurrent with hydrogel formation, a local aqueous reaction environment will be formed within the microcapsules.

A number of photo-initiators or photo-inducers are consistent with the disclosure herein. Exemplary photo-initiators include Type I photo-initiators, such as Igracure 2959 or lithium phenyl-2,4,6-trimethylbenzoylphosphinate (LAP, Sigma Aldrich, cat. no. 900889-1G). Other photoinducers responsive to electromagnetic radiation such as visible light, near ultraviolet light or ultraviolet light. Are consistent with the disclosure herein.

Upon induction of the free-radical forming agent, the hydrogel precursor monomer or other subunits are induced to form crosslinking bonds causing formation of the hydrogel shell at the exterior of the microcapsule.

However, off-target free-radical formation, such as that which occurs in the carrier in the vicinity of the carrier-droplet border or elsewhere in the carrier or in the pre-microcapsule droplet, may be sufficient to transmit a chemical stress into the local aqueous reaction environment so as to negatively impact cell viability or microcapsule content stability.

Accordingly, a common theme of many of the approaches disclosed herein for preserving microcapsule content integrity is to induce crosslinking without concomitant accumulation of reactive chemical stress transmitting molecules in the hydrogel microcapsule local aqueous reaction environment, or the microcapsule core precursor.

In some cases microcapsule populations arising from these or related approaches harbor viable cells, active biomolecules, or biomolecule effective concentrations at a frequency of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% of the frequency of that in the emulsion from which the microcapsules were generated.

A number of exemplary approaches for accomplishing this are presented below.

### Displacement

Some approaches to protection of hydrogel microcapsule local aqueous reaction environment comprise displacing potentially reactive chemical stress transmitting molecules from the droplet or droplet carrier phase or both prior to inducing crosslinking of the hydrogel microcapsule shell. This is accomplished, for example, by displacing oxygen from the droplet carrier phase prior to inducing crosslinking by flushing with an inert gas, such as Argon or other noble gas or other inert oxygen replacement.

This displacement of oxygen leaves the chemical stress without a carrier to deliver it to the aqueous core or to its precursor in an ATPS. Upon induction of hydrogel crosslinking, because there are few or no oxygen molecules in the microcapsule hydrogel precursor, reactive oxygen species are not generated in sufficient quantities to diffuse into the local aqueous reaction environment at levels sufficient to impact the sample such as to decrease cell viability. Similarly, reactive oxygen species harmful to samples are not generated in sufficient quantities in the local aqueous reaction environment by water soluble photo-inducers that have diffused into the aqueous environment.

In various embodiments of this approach, displacement is effected at the carrier, at the aqueous precursor to the emulsion droplets, or at the emulsion subsequent to its formation. Alternately or in combination, cells held in emulsion droplets are allowed to respire so as to locally deplete molecular oxygen from their droplet environment prior to crosslinking induction. An effect of each of these approaches is a reduction or elimination of molecular oxygen in the vicinity of free radical formation so as to prevent the transmission of chemical stresses to microcapsule contents such as cells.

Displacement is effected concurrent with or prior to crosslinking, and may be effected prior to, concurrent with or subsequent to emulsion generation in the carrier.

Displacement is effected, for example, by flushing with a noble gas or other stable gas, such as Argon, or even Helium. Neon, Krypton, Xenon, Radon, or molecular Nitrogen gas. Carbon dioxide, or other gas suitable for replacing a source of reactivity such as oxidative stress, for example Oxygen.

A number of carriers are suitable for Oxygen gas displacement, such as HFE7500 engineered fluid (available commercially from 3M), or HFE 7100, HT55, HT135, FC40, mineral oil or other carrier fluid. Information on O2 solubility in oil carriers is available, for example in the Journal of Fluorine Chemistry 125 (2004) 1325-1329, and in Journal of Fluorine Chemistry, 9 (1977)137-146, or is otherwise available or known to one of skill in the art. Similarly, approaches and conditions for flushing with a noble gas or other stable gas are known in the art or readily developed by the skilled practitioner.

In various cases flushing such as Argon flushing achieves actual clearance of at least 99%, at least 98%, at least 97%, at least 96%, at least 95%, at least 90%, at least 85%, at least 80%, at least 75%, at least 70%, at least 600%, at least 50%, at least 40%, at least 30%, at least 20%, or at least 10%/0 of the reactive target such as oxygen from microcapsule precursors.

In various cases flushing such as Argon flushing achieves effective clearance of at least 99%, at least 98%, at least 97%, at least 96%, at least 95%, at least 90%, at least 85%, at least 80%, at least 75%, at least 70%, at least 60%, at least 50%, at least 40%/6, at least 30%, at least 20%, or at least 10% of the reactive target such as oxygen from microcapsule precursors.

Accordingly, compositions consistent with the disclosure hereon comprise O₂ depleted HFE7500 engineered fluid, or HFE 7100, HT55, HT135, FC40, mineral oil or other carrier fluid, as well as Ar flushed HFE7500 engineered fluid, or HFE 7100, HT55, HT35, FC40, mineral oil or other carrier fluid, N2 flushed HFE7500 engineered fluid, or HFE 7100, HT55, HT135, FC40, mineral oil or other carrier fluid, or other noble gas flushed HFE7500 engineered fluid, or HFE 7100, HT55, HT135, FC40, mineral oil or other carrier fluid.

In some cases microcapsule populations harbor viable cells, active biomolecules, or biomolecule effective concentrations at a frequency of at least 99%, at least 98%, at least 97%, at least 96%, at least 95%, at least 90%, at least 85%, at least 80%, at least 75%, at least 70/o, at least 60%, at least 50%, at least 40%, at least 30%, at least 20%, or at least 10% of the frequency of that in the emulsion from which the microcapsules were generated.

Any of these compositions or compositions exhibiting similar characteristics may constitute a carrier for an emulsion, such as an emulsion comprising modified hexose or pentose hydrogel precursors. Exemplary precursors comprise methacryloyl-modified dextran, acryloyl-modified dextran, as well as a broad range of similarly modified hexose or pentose sugars, modified with any one of a broad range of moieties suitable for inducible crosslinking. Similarly, such an emulsion may in some cases comprise a sample subdivision, such as a cell, a population of cells, or other biochemically fragile constituent.

Any of these compositions may constitute a carrier for an emulsion, such as an emulsion comprising microcapsule precursors or microcapsules. The microcapsules in some cases comprise a hydrogel shell and an aqueous core. The hydrogel may comprise crosslinked methacryloyl-modified dextran, acryloyl-modified dextran, as well as a broad range of similarly modified hexose or pentose sugars, modified with any one of a broad range of moieties suitable for inducible crosslinking. The hydrogel shell is in some cases readily degraded under physiological conditions such as conditions suitable for enzymatic degradation by, for example, dextranase or other carbohydrate degrading enzyme. The aqueous core may comprise, for example, a hexose or pentose sugar such as dextrose, and in some cases further comprises a sample subdivision, such as a cell, a population of cells, or other biochemically fragile constituent.

### Exclusion

Some approaches to protection of hydrogel microcapsule local aqueous reaction environment comprise excluding the source of the potentially reactive chemical stress from the microcapsule precursor droplets. Similar to the displacement approaches above, these approaches result in the reduction or elimination of chemical stress molecules in the local aqueous reaction environment. While the previous approaches limited the chemical stress molecules themselves, these approaches physically restrict the location of the chemical stress inducer.

In particular, these approaches comprise the use of a cross-linking inducer that is physically excluded from the local aqueous reaction environment. For example, water-insoluble or oil-carrier soluble radical inducers, such as water-insoluble or oil-carrier soluble photo-inducers, can be used in place of water-soluble photo-inducers Water-insoluble or oil-carrier soluble radical inducers may be distributed throughout the carrier but will only contact the microcapsule precursor at its perimeter. Accordingly, free radicals such as free oxygen radicals or other chemical stress molecules will contact the microcapsule precursor droplet at its edge rather than throughout its volume. Crosslinking may occur at the hydrogel precursor monomer or other subunits to form crosslinking bonds causing formation of the hydrogel shell at the exterior of the microcapsule. However, free-radical formation does not occur in the interior of the local aqueous reaction environment, and external free radicals such as free oxygen radicals or other chemical stress molecules may be exhausted or extinguished in inducing crosslinking at the hydrogel perimeter, such that the local aqueous reaction environment, including the cell or other sample subdivision, is protected from chemical stress.

A number of water-insoluble or oil-carrier soluble radical inducers are known in the art and consistent with the disclosure herein. Generally, such inducers are soluble in the carrier, and generate radicals or other chemical moieties that diffuse or otherwise enter into emulsion droplets. However, the inducers themselves do not or do not substantially diffuse into the emulsion droplets, such that the radicals or other chemical moieties enter the emulsion droplets from the droplet exterior rather than being uniformly generated throughout the emulsion droplet interior volume. Accordingly, the radicals or other chemical moieties are likely to encounter the emulsion droplet constituents amassed at the droplet-carrier border, such as relatively hydrophobic hydrogel precursors, rather than core precursor constituents. Crosslinking is effected by the generation of chemically reactive moieties, but these moieties contact microcapsule precursors at their perimeter rather than permeating the volume as a whole. Accordingly, chemically reactive moieties are likely to exhaust themselves in crosslinking reactions to form hydrogel shell rather than penetrating to the interior of the microcapsule precursor to access the aqueous core or the sample.

In some cases, chemical reactive moieties exhibit an effective concentration at the center of a microcapsule precursor which is no more than 1%, no more than 2%, no more than 3%, no more than 4%, no more than 5%, no more than 10%, no more than 15%, no more than 20%, no more than 25%, no more than 30%, no more than 50%, or no more than 75% that of the perimeter of the microcapsule precursor.

In some cases microcapsule populations harbor viable cells, active biomolecules, or biomolecule effective concentrations at a frequency of at least 99%, at least 98%, at least 97%, at least 96%, at least 95%, at least 90° 0, at least 85%, at least 80%, at least 75%, at least 70%, at least 60%, at least 50%, at least 40%, at least 30%, at least 20%, or at least 10% of the frequency of that in the emulsion from which the microcapsules were generated.

Some exemplary carrier soluble inducers include Diphenyl(2,4,6-trimethylbenzoyl)phosphine oxide (TPO), Phenylbis(2,4,6-trimethylbenzoyl)phosphine oxide (BAPO), 9,10-Phenanthrenequnone, (PheQui), 4,4′-Bis(diethylamino)benzophenone (BABPhe), Methyl benzoylformate (MeBForm), among others. A carrier composition comprising one or more of the above inducers or another carrier-soluble inducer may further comprise one or more synergistic inducer supplement, such as EHDAB, TMP, or other synergistic amine or other synergistic molecule. Alternately. or in combination, two photon initiators may be used.

Accordingly, some compositions disclosed herein comprise one or more of Diphenyl(2,4,6-trimethylbenzoyl)phosphine oxide (TPO), Lithium phenyl-2,4,6-trimethylbenzoylphosphinate (LAP), Phenylbis(2,4,6-trimethylbenzoyl)phosphine oxide (BAPO), 9,10-Phenanthrenequinone, (PheQui), 4,4′-Bis(diethylamino)benzophenone (BABPhe), Methyl benzoylformate (MeBForm), among others, in combination with a hydrophobic carrier such as HFE7500 engineered fluid, or HFE 7100, HT55, HT135, FC40, mineral oil or other carrier fluid, and in some case further comprising an aqueous emulsion of droplets comprising microcapsule precursors such as a hexose or pentose sugar such as dextrose, and in some cases further comprises a sample subdivision, such as a cell, a population of cells, or other biochemically fragile constituent. Some compositions disclosed herein comprise one or more of Diphenyl(2,4,6-trimethylbenzoyl)phosphine oxide (TPO), Lithium phenyl-2,4,6-trimethylbenzoylphosphinate (LAP), Phenylbis(2,4,6-trimethylbenzoyl)phosphine oxide (BAPO), 9,10-Phenanthrenequinone, (PheQui), 4,4′-Bis(diethylamino)benzophenone (BABPhe), Methyl benzoylformate (MeBForm), among others, in combination with a hydrophobic carrier such as HFE7500 engineered fluid, or HFE 7100, HT55, HT135, FC40, mineral oil or other carrier fluid, and in some case further comprising a microcapsule population, for example comprising particles having hydrogel shells that are readily degradable under physiological conditions by, for example, a dextranase, and aqueous interiors, in some cases further comprises a sample subdivision, such as a cell, a population of cells, or other biochemically fragile constituent.

### Cell-Protecting Agents

Through both exclusion and displacement, chemical stress such as free radical formation is induced and used to drive hydrogel formation, but the impact on the local aqueous reaction environment is reduced because the chemical stress carrier molecules are either displaced from the local aqueous reaction environment, or are only induced in the carrier such that they only or primarily contact the local aqueous reaction environment at the hydrogel forming perimeter.

Also disclosed herein are methods, systems and compositions that reduce or eliminate the impact on the local aqueous reaction environment of chemical moieties by employing molecules that act as scavengers, protecting agents or antioxidants in the emulsion droplet or the microcapsule precursor to protect cells or other sample subdivision contents. That is, these protective agents blunt the effect of any chemically reactive moieties in the vicinity of the sample subdivision contents.

These methods, systems and compositions are practiced in isolation or in combination with Displacement or Exclusion approaches, or both, such as those disclosed above.

Scavenger, protectant or antioxidant approaches disclosed herein protect the cells or sample subdivisions, but often do not preclude hydrogel formation at the droplet perimeter so as to form microcapsules. Scavengers, protectants or antioxidants react with chemical moieties that induce crosslinking but that may in some cases harm cells or other sample subdivisions, so as to reduce the impact on cells or sample subdivisions without precluding hydrogel formations. Exemplary scavengers, protectants or antioxidants include DTT, Ascorbic acid, Mannitol, Inositol, H0726, N-acetyl-L-cysteine, Sorbitol or other moieties that may soften the impact of a chemical crosslinking inducer on cells or sample subdivisions. In some cases, the aqueous core precursor, such as dextran, is selected to have a scavenger, protectant or antioxidant effect so as to protect the cell or other sample subdivision contents. Scavengers, protectants or other antioxidants are added to the emulsion precursor prior to, concurrently with, or subsequent to addition of the cell or other subdivision, and prior to, concurrently with, or subsequent to emulsion formation.

Accordingly, some compositions disclosed herein comprise emulsions wherein the droplets or carrier or both droplets and carrier comprise a chemically reactive moiety protectant such as one or more of DTT, Ascorbic acid, Mannitol, Inositol, H0726, N-acetyl-L-cysteine, Sorbitol or other moieties that may soften the impact of a chemical crosslinking inducer on cells or sample subdivisions. Such compositions may further or alternately comprise one or more of Diphenyl(2,4,6-trimethylbenzoyl)phosphine oxide (TPO). Lithium phenyl-2,4,6-trimethylbenzoylphosphinate (LAP), Phenylbis(2,4,6-trimethylbenzoyl)phosphine oxide (BAPO), 9,10-Phenanthrenequinone, (PheQui), 4,4′-Bis(diethylamino)benzophenone (BABPhe), Methyl benzoylformate (MeBForm), among others. Alternately or in combination, such compositions may comprise a hydrophobic carrier such as HFE7500 engineered fluid (available commercially from 3M), or HFE 7100, HT55, HT135. FC40, mineral oil or other carrier fluid, optionally evacuated as disclosed herein or otherwise known in the art, and in some cases further comprises a sample subdivision, such as a cell, a population of cells, or other biochemically fragile constituent.

In some cases, chemical reactive moieties are absorbed, scavenged or quenched such that they exhibit an effective concentration at the center of a microcapsule precursor which is no more than 1%, no more than 2%, no more than 3%, no more than 4%, no more than 5%, no more than 10%, no more than 15%, no more than 20%, no more than 25%, no more than 30%, no more than 50%, or no more than 75% that of the perimeter of the microcapsule precursor.

In some cases microcapsule populations arising from these or related approaches harbor viable cells, active biomolecules, or biomolecule effective concentrations at a frequency of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% of the frequency of that in the emulsion from which the microcapsules were generated.

### Radical-Free Crosslinking

Some approaches to protection of hydrogel microcapsule local aqueous reaction environment comprise avoiding the generation of potentially reactive chemical stress. Similar to the displacement or exclusion approaches above, these approaches result in the prevention of accumulation of chemical stress molecules in the local aqueous reaction environment. The previous approaches limited the chemical stress molecules themselves, or physically restrict the location of the chemical stress inducer, or scavenge chemical stress molecules from the emulsion, microcapsule precursor interior, or microcapsule interior. In contrast, radical free approaches effect hydrogel formation without generation of potentially reactive chemical stress.

Some such approaches use an alternative chemical initiator, such as a crosslinking molecule, for example a crosslinking molecule having distinct functional groups that may bind hydrogel constituents, rather than free-radicals or oxidative stress to induce hydrogel crosslinking, such as at methacryloyl or acryloyl moieties on a pentose or hexose such as dextran. To facilitate these approaches, methacryloyl or acryloyl moieties may be present on monomers at a higher frequency than in compositions relating to oxidative stress induced crosslinking, such as in 60:40 DexMAB, which as up to 6× higher concentration of methacryloyl or acryloyl moieties relative to 10:90 DexMAB or other compositions used herein. Alternate cross-linking moieties harboring other double-bond-carrying functional groups for radical-free cross-linking, such as maleimide-modified dextran, may be used to effect hydrogel crosslinking.

A number of agents are known in the art to be both sufficient for inducing crosslinking and biocompatible with microcapsule payloads such as cells. An exemplary crosslinking agent is dithiothreitol (DTT), while alternatives harboring multiple functional moieties are also contemplated.

Representative approaches for radical-free hydrogel formation include Michael reactions, such as thia-Michaels reactions. These reactions comprise the addition of a nucleophile such as an enolate of a ketone or aldehyde to an α,β-unsaturated carbonyl compound at the p carbon. Similarly, Diels-Alders reactions, reactions employing click-chemistry, such as copper catalyzed alkyne-azide reactions or copper-free alkyne reactions, or even noncovalent hydrogel formation such as via binding partner assembly of hydrogels (for example, biotin-avidin or streptavidin mediated hydrogel formation). Some approaches to hydrogel formation comprise ring-opening metathesis or disulfide formation.

Crosslinking is in some cases effected by subjecting the microcapsule precursor and the cross-linking agent to an alkaline pH, such as a pH of at least 8, at least 8.5, at least 9, at least 9.5, at least 10 or greater than 10.

Common features of these approaches include the formation of microcapsule shells without the use of free radicals or reactive oxygen species to effect hydrogel formation. Accordingly, without being bound by theory, these approaches are likely to reduce the exposure of a cell or other sample subdivision to harmful chemical moieties.

However, these approaches need not be used in isolation or as strict alternatives to other hydrogel forming approaches. That is, radical-free or photo-induction free approaches for hydrogel formation as disclosed herein are in some cases used in combination with rather than as an alternative to approaches to hydrogel formation that comprise generation of free-radicals or harsh chemical moieties.

One benefit of combining approaches is that it allows one to reduce the effective concentration of the chemical stress moiety to which the cell or other sample subdivision is exposed. This is accomplished by reducing the time, concentration of reagents used, or intensity of activation of the chemical stress moiety such as a free radical or reactive oxygen species. Combining approaches may also allow one to achieve Displacement because the radical-based cross-linking may be performed after transferring capsules into an environment with lower oxygen solubility. For example, performing an initial gelation step using DTT in an emulsion of fluorinated oil (in which oxygen is highly soluble) may be followed by transferring the microcapsules to an aqueous solution and subjecting them to further hardening using LAP-mediated radical formation. As oxygen is less soluble in water than in the emulsion carrier, the molecular oxygen is effectively displaced prior to radical photo-induction. Without being bound by theory, in these combination approaches the hydrogel induction effects of radical and non-radical induction approaches are additive, but the harm to microcapsule contents is not.

Accordingly, some compositions disclosed herein comprise emulsion of microcapsule precursors comprising hydrogel precursors tethered to binding agent pairs. A number of binding agent pairs are consistent with the disclosure herein, such as ligand-receptor pairs or antigen-antibody pairs, among others. Exemplary binding agent pairs are biotin-avidin or biotin-streptavidin pairs. These emulsions are consistent with a broad range of carriers, such as the carriers mentioned herein or otherwise compatible with the disclosure.

In some cases, these emulsions further comprise aqueous core precursors such as dextran or other core components disclosed herein, and may further comprise a cell or cell population or other sample subdivision.

The emulsions variously comprise hydrophilic or a hydrophobic free radical inducer, situated in the emulsion droplets or in the carrier. Similarly, in various cases the droplets or the carrier is evacuated of molecular oxygen or other chemical stress moiety precursor. Consistent with the disclosure herein, combinatorial approaches using one or more of displacement, exclusion, cell-protection or scavenging and radical-free hydrogel formation may be used.

In some cases microcapsule populations harbor viable cells, active biomolecules, or biomolecule effective concentrations at a frequency of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% of the frequency of that in the emulsion from which the microcapsules were generated

### Microcapsule Populations

Through practice of the methods and use of the compositions disclosed herein, compositions are generated comprising microcapsule populations harboring contents such as biological contents therein. The contents are in some cases biochemically fragile, such as viable cells, cell organelles such as nuclei, ribosomes, assembled transcription or translation complexes, metabolically active mitochondria, plastids, proteosomes, vacuoles, or other organelles, or viral particles or proteins or protein complexes or other biological contents that are vulnerable to oxygen free radicals or other crosslinking moieties, but that may nonetheless be biochemically robust or physiologically operational. That is, mitochondria remain capable of transcription, translation and respiration, for example, nuclei continue to support transcription, and chloroplasts remain capable of transcription, translation and photosynthesis.

The contents may comprise cells, such as primary cells—that is, cells extracted or obtained from a primary patient or other sample. These cells may comprise eukaryotic or human cells. These cell types are generally more challenging to work with than are cultured cell lines, which are generally selected for viability and proliferative capacity and are therefore more readily encapsulated even under relatively harsh conditions.

Primary cells may be obtained from a number of sources, such as environmental sources, patient blood or other fluid samples, tissue samples such as infected tissue samples, diseased tissue samples such as samples harboring beta amyloid plaques, tumor samples, or other samples comprising a diversity of cells that may exhibit differential viability in response to a harsher encapsulation process. Primary cells may be prokaryotic or eukaryotic, and may be healthy human cells, pathogen cells, tumor cells, senescent cells or other cell types.

Dissociated tumor samples, for example, harbor a diversity of cell types and a diversity of genotypes resulting from accumulated and in some cases oncogenic mutations that arise or trigger tumor growth. Tumors often comprise a diversity of cell populations, most of which are quiescent and may be senescent, while only a minority of the cells constitute tumor stem cells that drive tumor or cancer proliferation. Failure to isolate the full diversity of cell types in a sample such as this may have a substantial negative impact on the utility of the downstream analysis.

Similarly, samples from individuals suffering from an infectious disease may harbor a large diversity of xenic cell or viral types. This is also particularly true of environmental or gastrointestinal samples, but is true of a number of sample types. By capturing the full diversity of cell and viral types in the sample, one may more confidently use the subsequent microcapsule population to assay for candidate pathogens. Similarly, by exchanging growth media, one is more likely to identify conditions under which known or unknown pathogens may proliferate, thereby facilitating downstream analysis such as nucleic acid sequencing.

Environmental samples, such as marine water, sewage, soil, and air may harbor cells or cell communities producing medically, environmentally, and industrially relevant compounds, such as antibiotics, antibiotic resistance genes, biofuels, enzymes, and other biotechnological products. The species of interest in such samples, including their susceptibility to biologically harsh conditions including damage by radical and reactive oxygen species, are typically unknown a priori. By capturing the full diversity of cell and viral types in the sample, one may more confidently use the subsequent microcapsule population to assay for biological activities of interest, such as desired compound production. In experiments involving the culturing of encapsulated microbes by placing them in the native source environment, capsules offer the benefit of preventing fast growers from overtaking the culture because the expansion of a given cell is limited to the volume of the capsule.

Cells may not exhibit an impact on viability as a result of a microcapsule generation process. That is, the cell populations may exhibit viability rates that are comparable to those of cell populations prior to encapsulation. For example, encapsulated cell populations may exhibit viability rates that are at least 10%, 20%, 30%, 40%, 50%, 60%, 70%, 80%, 90%, 95% or greater than 95% that of the cells prior to microencapsulation.

A number of parameters may be used to quantify success of the methods herein in “gently” encapsulating sample partitions such as cells, viral particles or biochemical moieties.

Cells may not exhibit a transcriptional impact of the microcapsule generation process. That is, the cells may not exhibit chemical stress-induced changes to transcription profiles or metabolic activity, while redox-sensitive organelles such as mitochondria or plastids may not exhibit an impact on their redox-sensitive proton transport or light or dark reaction photosynthesis activities. For example, encapsulated cells may exhibit redox-sensitive proton transport or light or dark reaction photosynthesis activities that are at least 10%, 20%, 30%, 40%, 50%, 60%, 70%, 80%, 90%, 95% or greater than 95% that of the cells prior to microencapsulation.

Cells may not exhibit a proliferative impact of the microcapsule generation process. That is, the cells may not exhibit chemical stress-induced changes to mitotic proliferation rates. For example, encapsulated cells may exhibit proliferative rates that are at least 10%, 20%, 30%, 40%, 50%, 60%, 70%, 80%, 90%, 95% or greater than 95% that of the cells prior to microencapsulation.

Cells may not exhibit a metabolic impact of the microcapsule generation process. That is, the cells may not exhibit chemical stress-induced changes to metabolic activity. For example, encapsulated cells may exhibit metabolic rates that are at least 10%, 20%, 30%, 40%, 50%, 60%, 70%, 80%, 90%, 95% or greater than 95% that of the cells prior to microencapsulation.

Cells may not exhibit a free radical stress response as a result of the microcapsule generation process. That is, the cells may not exhibit chemical stress-induced changes to transcription or translation or protein accumulation of free radical or oxidative stress response factors such as superoxide dismutase or free radical scavenging enzymes or activation of stress-response signaling pathways. For example, encapsulated cells may exhibit free radical or oxidative stress response factor accumulation levels that differ from levels prior to encapsulation by no more than 50%, 40%, 30%, 20%, 10% or less than 10%.

Similarly, populations may be generated where the frequency or density of microcapsules harboring viable cells in the microcapsule population is reflective of the frequency or density of viable cells in the sample subdivisions are taken to generate the microcapsule population. In some cases, the frequency or density of microcapsules harboring viable cells in the microcapsule population is at least 10%, 20%, 30%, 40%, 50%, 60%, 70%, 80%, 90%, 95% or greater than 95% that of the frequency or density of viable cells in the sample prior to subdivision into microcapsule precursor droplets.

Alternately, in some cases populations may be generated where the frequency or density of microcapsules harboring viable cells in the microcapsule population is at least 10%, 20%, 30%, 40%, 50%, 60%, 70%, 80%, 90%, 95% or greater. In some cases, this frequency of microcapsules harboring viable cells is attained without enriching for microcapsules harboring viable cells.

Populations may be generated where the encapsulated cell populations are reflective of the cell types, relative cell numbers, or both relative cell types and numbers, of cells in the precursor sample. That is, in samples that comprise a diverse cell population, the cell diversity and relative proportions may be reflected in the numbers and proportions of cells encapsulated in the microcapsule population. This diversity is captured even when the cells of the sample exhibit differential viability, such that some cells of the sample are much more robust than others. In some prior art encapsulation approaches, cells may be encapsulated, but the process may select for only the most robust or durable cells among the sample, such that the population of encapsulated cells is not quantitatively or qualitatively reflective of the sample cell diversity prior to encapsulation. Accordingly, in some cases the microcapsule population comprises at least 10%, 20%, 30%, 40%, 50%, 60%, 70%, 80%, 90° %, 95% or greater than 95% up to 100% of the cell types present in the sample prior to encapsulation. Similarly, n some cases the microcapsule population comprises cell types at relative proportions that differ from the relative proportions of the sample by no more than 50%, 25%, 20%, 15%, 10%, 5% or less than 5%.

Taking as an example a sample comprising cells obtained from a disarticulated tumor, one sees through the practice of the approaches herein that both rare and abundant cells of the tumor are encapsulated, and that the microcapsule population does not exhibit a bias or does not exhibit a substantial bias toward durable cells over senescent, quiescent or near-apoptotic cells of the disarticulated tumor cell population.

Microcapsule populations may comprise a diversity of encapsulated cells that reflects a pre-encapsulation cell diversity, such that vulnerable cells are not differentially lost in the encapsulation process. In some cases, the encapsulated cell diversity is such that is at least 10%, 20%, 30%, 40%, 50%, 60%, 70%, 80%, 90%, 95% or greater of the cell types in the pre-encapsulation composition are represented in the microcapsule population. In some examples, the microcapsule population represents at least 10%, 20%, 30%, 40%, 50%, 60%, 70%, 80%, 90%, 95% or greater of the cell types of a composition comprising a disarticulated tumor.

By capturing a broad range of cells rather than selecting only for the healthiest, actively dividing or stress tolerant among a sample cell population, one may be more likely to encapsulate quiescent, stress-sensitive or otherwise difficult to culture cells from a sample population. Once encapsulated, one may subject the microcapsule population to a number of reagent replacement washes so as to identify reagent conditions that facilitate proliferation of such problematic cell populations.

Any of the populations above may comprise at least 50, at least 100, at least 200, at least 500, at least 1000, at least 2,000, at least 5,000, at least 10.000, at least 20,000, at least 50,000, at least 100.000, at least 200.000, at least 500,000, or at least 1,000,000 microcapsules.

A feature shared by many of the microcapsule populations herein is that, in addition to “gently” encapsulating their contents, they may “gently” release their contents. Release may be effected under physiological conditions, such as those suitable for enzyme activity. In particular, some microcapsule populations comprise hydrogel shells comprising an enzymatically degradable constituent as disclosed herein, for example a carbohydrate such as DexMAB, that is readily degraded by an enzyme such as a glycosidase (dextranase, in the case of DexMAB, though others may also be suitable). This degradation occurs under conditions that are “gentle” to the microcapsule contents, so as to release the contents without subjecting them to harsh physiological conditions, such that upon release microcapsule contents, there is an impact comparable to the impact cited above in this section, for example on cell viability, transcriptome accumulation levels, stress response, or differential cell viability.

Turning to the figures, one sees the following.

At FIG. 1, one sees a scheme for reagent replacement.

At FIG. 2, one sees Bright-field microscopy images showing K562 growth in SPCs generated by DTT-induced gelation (in emulsion) followed by emulsion-free photopolymerization in aqueous suspension. Randomly selected images show representative K-562 expansion to multicellular spheroids over six days.

At FIG. 3, one sees Bright-field microscopy image showing K562 cells in SPCs after six days in culture. Arrows indicate cells that failed to divide.

At FIG. 4, one sees a Bright Field microscopy image showing failure to form microcapsules able to survive 6 days under K562 growth conditions. DTT-induced gelation was used as the radical-free crosslinking strategy. Sufficient gelation was not allowed to occur by breaking the water-in-oil emulsion right after collection.

At FIG. 5 and FIG. 6, one sees Bright Field microscopy images showing stably formed microcapsules, some of which harbored proliferating K5623 cells at 6 days under K562 growth conditions. The microcapsule population harbors cells at a rate comparable to the proportion expected from the cell input concentration. DTT-induced gelation was used as the radical-free crosslinking strategy. Sufficient gelation was allowed to occur by incubating the collected emulsion for 45 min at room temperature (FIG. 5) or +4° C. (FIG. 6) breaking it and transferring capsules into an aqueous phase.

At FIG. 7, one sees Bright Field microscopy images showing stably formed microcapsules in oil, generated using BAPO in carrier solution augmented with EHDAB. The microcapsule population is made without introducing the photo-inducer into the emulsion droplets.

At FIG. 8, one sees Bright Field microscopy images showing stably formed microcapsules resuspended in aqueous solution, generated using BAPO in carrier solution. The microcapsule population is made without introducing the photo-inducer into the emulsion droplets.

At FIG. 9, one sees Bright Field microscopy images showing stably formed microcapsules resuspended in aqueous solution, generated using BAPO in carrier solution augmented with EHDAB. The microcapsule population is made without introducing the photo-inducer into the emulsion droplets.

At FIG. 10 one sees a time course of K562 cell growth in a microcapsule population generated in the absence (left column, Control) and presence (right column, Arg-flushed) of Argon flushing associated with oxidative stress mediated microcapsule formation. Images were taken at time 0, at 38 hours, at 48 hours, at 68 hours and at 7 days. One sees a substantially greater number of SPCs harboring proliferating cell colonies in the Argon treated SPC population than in the control. This demonstrates the protective effect of excluding oxidative stress conducting moieties from droplet cores in proximity to biological material such as cells, during hydrogel formation such as through oxidative stress inducing UV, near ultraviolet visible or other visible light administration.

At FIG. 11, one sees a time course of cell proliferation among cells encapsulated in 10:90 DexMab in the presence of DTT as cell-protecting agent using radical-based cross-linking (top) and in 60.40 DexMab in the absence of induced oxidative stress ensured by use of a radical-free cross-linking strategy (bottom). Cell proliferation is observed over a 7 day time course in single day intervals. FIG. 11 demonstrates that DTT was able to protect cells during oxidative stress mediated microgel formation such that they exhibited survival rates comparable to those of cells encapsulated using an oxidative stress free approach.

### Definitions

As used herein, the term “about” in the context of a number refers to a range spanning 10% below the number to 10% above the number, while in the context of a range, the term refers to an extended range spanning from 10% below the listed lower limit to 10% above the upper listed limit.

As used herein, the phrase “at least one of” in the context of a group A. B, and C, for example, refers to sets including A, alone or with unlisted factors, B, alone or with unlisted factors, C, alone or with unlisted factors, A and B, alone or with unlisted factors, A and C, alone or with unlisted factors, B and C, alone or with unlisted factors, or A, B, and C, alone or with unlisted factors.

As used herein, the phrases “at least” or “no greater than” in the context of a following list of numbers are understood to apply distributively throughout the list, rather than only to the initial value of the list.

### NUMBERED EMBODIMENTS

The disclosure is further understood in light of the following numbered embodiments, which are understood to be combinable with one another and with elements recited elsewhere herein
1. A method of crosslinking a capsule shell while ameliorating an impact of a chemical stress on an interior space of the capsule, the method comprising preventing a chemical stress moiety from being active in an interior space of the capsule, and inducing radical formation by a crosslinking initiator dissolved in the capsule. 2. The method of any listed embodiment, such as number 1, wherein the chemical stress is an oxidative stress. 3. The method of any listed embodiment, such as number 1, wherein the chemical stress is a free radical stress 4. The method of any listed embodiment, such as number 1, wherein preventing a chemical stress moiety from being active in the interior space of the capsule comprises displacing dissolved oxygen molecules from the interior space of the capsule. 5. The method of any listed embodiment, such as number 1, wherein the capsule comprises a modified polysaccharide backbone. 6. The method of any listed embodiment, such as number 5, wherein the modified polysaccharide backbone comprises a acryloyl moiety. 7. The method of any listed embodiment, such as number 5, wherein the modified polysaccharide backbone comprises a methacryloyl moiety. 8. The method of any listed embodiment, such as number 5, wherein the modified polysaccharide backbone comprises a conjugated diene. 9. The method of any listed embodiment, such as number 5, wherein the modified polysaccharide backbone comprises a substituted diene. 10. The method of any listed embodiment, such as number 5, wherein the modified polysaccharide backbone comprises an azide 11. The method ofany listed embodiment, such as number 5, wherein the modified polysaccharide backbone comprises an alkyne. 12. The method of any listed embodiment, such as number 5, wherein the modified polysaccharide backbone comprises a nitrone. 13. The method of any listed embodiment, such as number 5, wherein the modified polysaccharide backbone comprises a maleimide. 14. The method of any listed embodiment, such as number 5, wherein the modified polysaccharide backbone comprises biotin. 15. The method of any listed embodiment, such as number 5, wherein the modified polysaccharide backbone comprises avidin. 16. The method of any listed embodiment, such as number 5, wherein the modified polysaccharide backbone comprises streptavidin. 17. The method of any listed embodiment, such as number 5, wherein the modified polysaccharide backbone comprises a cyclic olefin. 18. The method of any listed embodiment, such as number 5, wherein the modified polysaccharide backbone comprises a thiol group. 19. The method of any listed embodiment, such as number 1, wherein displacing dissolved oxygen molecules comprises flushing the interior space using a gas that excludes oxygen. 20 The method of any listed embodiment, such as number 19, wherein the gas comprises nitrogen molecules. 21. The method of any listed embodiment, such as number 19, wherein the gas comprises noble gas molecules. 22. The method of any listed embodiment, such as number 19, wherein the gas comprises argon molecules. 23. The method of any one of any listed embodiment, such as numbers 1-22, wherein the crosslinking initiator is photoactivatable. 24 The method of any one of any listed embodiment, such as numbers 1-22, wherein the crosslinking initiator induces radical crosslinking in response to visible or ultraviolet light. 25. The method of any listed embodiment, such as number 1, wherein the oxidative stress moiety comprises a water-insoluble crosslinking initiator. 26. The method of any listed embodiment, such as number 25, wherein water-insoluble crosslinking initiator is located in a hydrophobic carrier liquid that harbors a precursor to the capsule prior to crosslinking. 27. The method of any listed embodiment, such as number 26, wherein the water-insoluble crosslinking initiator is photoactivatable. 28. The method of any listed embodiment, such as number 26, wherein the water-insoluble crosslinking initiator generates radicals. 29. The method of any listed embodiment, such as number 28, wherein the radicals do not reach the interior space of the capsule. 30. The method of any listed embodiment, such as number 29, wherein the water-insoluble crosslinking initiator is present at a concentration insufficient to allow radicals to reach the interior space of the capsule. 31. The method of any listed embodiment, such as number 29, wherein capsule shell is present at a thickness insufficient to allow radicals to reach the interior space of the capsule. 32. The method of any listed embodiment, such as number 1, wherein preventing an oxidative stress moiety from being active in interior space of the capsule comprises crosslinking the capsule without generating oxidative radicals. 33. The method of any listed embodiment, such as number 32, wherein a capsule shell precursor comprises a double bond 34. The method of any listed embodiment, such as number 33, wherein the capsule shell precursor comprises a sugar. 35. The method of any listed embodiment, such as number 33, wherein the capsule shell precursor comprises an acryloyl modified sugar. 36. The method of any listed embodiment, such as number 33, wherein the capsule shell precursor comprises a methacryloyl modified sugar. 37. The method of any listed embodiment, such as number 33, wherein the capsule shell precursor comprises an acryloyl modified dextran. 38. The method of any listed embodiment, such as number 33, wherein the capsule shell precursor comprises a methacryloyl modified dextran. 39. The method of any listed embodiment, such as number 33, wherein the capsule shell precursor comprises a maleimide modified sugar. 40. The method of any listed embodiment, such as number 33, wherein the capsule shell precursor comprises a maleimide modified dextran. 41. The method of any one of any listed embodiment, such as numbers 32-40, comprising contacting the capsule shell precursor to a thia-Michael reagent. 42. The method of any one of any listed embodiment, such as numbers 32-40, comprising contacting the capsule shell precursor to DTT. 43. The method of any one of any listed embodiment, such as numbers 1-37, wherein the interior space comprises a viable cell. 44. The method of any listed embodiment, such as number 43, wherein the cell is a eukaryotic cell. 45. The method of any listed embodiment, such as number 43, wherein the cell is a primary cell. 46. The method of any listed embodiment, such as number 43, wherein the cell is a stress-sensitive cell. 47. The method of any listed embodiment, such as number 43, wherein the cell exhibits a transcription profile that differs by no more than 10% from an unencapsulated cell of a sample the encapsulated cell is drawn from. 48. The method of any listed embodiment, such as number 43, wherein the cell exhibits a transcription profile that differs by no more than 1% from an unencapsulated cell of a sample the encapsulated cell is drawn from. 49. The method of any listed embodiment, such as number 43, wherein the cell exhibits a stress response level that differs by no more than 10% from an unencapsulated cell of a sample the encapsulated cell is drawn from. 50. The method of any listed embodiment, such as number 43, comprising culturing the viable cell. 51. An emulsion comprising a carrier comprising a crosslinking initiator and droplets comprising a shell precursor, a core precursor and a viable cell, wherein the droplets are substantially free of exogenous oxygen radicals. 52. The emulsion of any listed embodiment, such as number 51, wherein the carrier and the crosslinking initiator are hydrophobic. 53. The emulsion of any listed embodiment, such as number 51, wherein the carrier and the crosslinking initiator are lipophilic. 54. The emulsion of any listed embodiment, such as number 51, wherein the carrier is a fluorinated hydrocarbon and the crosslinking initiator is fluorophilic. 55 The emulsion of any listed embodiment, such as number 51, wherein the crosslinking initiator is not water soluble. 56. The emulsion of any one of any listed embodiment, such as numbers 51-55, wherein the droplets are substantially free of oxygen radicals arising from the crosslinking initiator. 57. A microcapsule population comprising at least 100 microcapsules, each microcapsule comprising a crosslinked shell and an aqueous interior, each aqueous interior comprising a viable cell such that the microcapsule population comprises at least 100 cells. 58. The microcapsule population of any listed embodiment, such as number 57, wherein a cell of the microcapsule population differs in a parameter by no more than 50% from a corresponding cell of a microcapsule precursor sample population. 59. The microcapsule population of any listed embodiment, such as number 58, wherein a cell of the microcapsule population differs in a parameter by no more than 25% from a corresponding cell of a microcapsule precursor sample population. 60. The microcapsule population of any listed embodiment, such as number 58, wherein a cell of the microcapsule population differs in a parameter by no more than 10% from a corresponding cell of a microcapsule precursor sample population. 61. The microcapsule population of any listed embodiment, such as number 58, wherein a cell of the microcapsule population differs in a parameter by no more than 5% from a corresponding cell of a microcapsule precursor sample population. 62. The microcapsule population of any one of any listed embodiment, such as numbers 58-61, wherein the parameter comprises transcript accumulation pattern. 63. The microcapsule population of any one of any listed embodiment, such as numbers 58-61, wherein the parameter comprises proteome accumulation pattern. 64. The microcapsule population of any one of any listed embodiment, such as numbers 58-61, wherein the parameter comprises stress response activity. 65. The microcapsule population of any one of any listed embodiment, such as numbers 58-61, wherein the parameter comprises growth rate. 66. The microcapsule population of any one of any listed embodiment, such as numbers 58-61, wherein the parameter comprises viability. 67. The microcapsule population of any one of any listed embodiment, such as numbers 58-61, wherein the parameter comprises cell viability dye fluorescence. 68. The microcapsule population of any one of any listed embodiment, such as numbers 58-61, wherein the parameter comprises trypan blue staining. 69. The microcapsule population of any one of any listed embodiment, such as numbers 58-61, wherein the parameter comprises cell motility. 70. The microcapsule population of any listed embodiment, such as number 57, wherein the at least 100 encapsulated cells differ in proportional cell identity by no more than 25% from a population of cells selected from a microcapsule sample precursor population. 71. The microcapsule population of any listed embodiment, such as number 57, wherein the at least 100 encapsulated cells differ in proportional cell identity by no more than 10% from a population of cells selected from a microcapsule sample precursor population. 72. The microcapsule population of any listed embodiment, such as number 57, wherein the at least 100 encapsulated cells differ in proportional cell identity by no more than 5% from a population of cells selected from a microcapsule sample precursor population. 73. The microcapsule population of any one of any listed embodiment, such as numbers 57-72, wherein the microcapsule population is degradable under physiological conditions. 74. The microcapsule population of any one of any listed embodiment, such as numbers 57-72, wherein the microcapsule population is enzymatically degradable. 75. The microcapsule population of any one of any listed embodiment, such as numbers 57-72, wherein the microcapsule population is degradable by treatment with a dextranase. 76. A method of crosslinking a droplet perimeter while ameliorating an impact of a chemical stress on an interior volume of the droplet, the method comprising preventing the chemical stress moiety from being active in the interior volume of the droplet, and allowing the chemical stress moiety to mediate crosslinking at the droplet perimeter. 77. The method of any listed embodiment, such as number 76, wherein the chemical stress is oxidative stress. 78. The method of any listed embodiment, such as number 76, wherein the chemical stress is a free radical stress. 79. The method of any listed embodiment, such as number 76, wherein the interior volume comprises a biomolecule. 80. The method of any listed embodiment, such as number 76, wherein the interior volume comprises a living cell. 81. The method of any listed embodiment, such as number 76, wherein the interior volume comprises a living eukaryotic cell. 82. The method of any listed embodiment, such as number 76, wherein the interior volume comprises a chemical stress moiety quencher. 83. The method of any listed embodiment, such as number 82, wherein the quencher comprises a redox modulator. 84. The method of any listed embodiment, such as number 82, wherein the quencher comprises DTT. 85. The method of any listed embodiment, such as number 76, wherein the interior volume comprises a chemical stress moiety protectant. 86. The method of any listed embodiment, such as number 85, wherein the protectant comprises a redox modulator. 87. The method of any listed embodiment, such as number 85, wherein the protectant comprises DTT. 88. The method of any listed embodiment, such as number 76, wherein the interior volume comprises a chemical stress moiety excluder. 89. The method of any listed embodiment, such as number 85, wherein the excluder comprises a noble gas. 90. The method of any listed embodiment, such as number 85, wherein the excluder comprises Argon. 91. The method of any listed embodiment, such as number 85, wherein the excluder comprises nitrogen gas. 92. The method of any listed embodiment, such as number 85, wherein the excluder comprises carbon dioxide. 93. The method of any listed embodiment, such as number 85, wherein the excluder clears oxygen gas from the interior volume. 94 The method of any listed embodiment, such as number 76, wherein preventing the chemical stress from being active in the interior of the droplet comprises quenching the chemical stress moiety. 95. The method of any listed embodiment, such as number 94, wherein quenching the chemical stress moiety comprises contacting the chemical stress moiety to a redox reagent. 96. The method of any listed embodiment, such as number 94, wherein quenching the chemical stress moiety comprises contacting the chemical stress moiety to DTT. 97. The method of any listed embodiment, such as number 94, wherein quenching comprises reducing the activity of the chemical stress moiety by at least 30%. 98. The method of any listed embodiment, such as number 94, wherein quenching comprises reducing the activity of the chemical stress moiety by at least 50%. 99. The method of any listed embodiment, such as number 94, wherein quenching comprises reducing the activity of the chemical stress moiety by at least 80%, 100. The method of any listed embodiment, such as number 94, wherein quenching comprises reducing the activity of the chemical stress moiety by at least 90%. 101. The method of any listed embodiment, such as number 94, wherein quenching comprises reducing the activity of the chemical stress moiety by at least 95%. The method of any listed embodiment, such as number 94, wherein quenching comprises reducing the effective concentration of the chemical stress moiety by at least 30%, 102. The method of any listed embodiment, such as number 94, wherein quenching comprises reducing the effective concentration of the chemical stress moiety by at least 50%, 103. The method of any listed embodiment, such as number 94, wherein quenching comprises reducing the effective concentration of the chemical stress moiety by at least 80%, 104. The method of any listed embodiment, such as number 94, wherein quenching comprises reducing the effective concentration of the chemical stress moiety by at least 90%. 105. The method of any listed embodiment, such as number 94, wherein quenching comprises reducing the effective concentration of the chemical stress moiety by at least 95%, 106. The method of any listed embodiment, such as number 76, wherein preventing the chemical stress from being active in the interior of the droplet comprises protecting against the chemical stress. 107. The method of any listed embodiment, such as number 106, wherein protecting against the chemical stress comprises contacting the chemical stress to a redox reagent. 108. The method of any listed embodiment, such as number 106, wherein protecting against the chemical stress comprises contacting the chemical stress to DTT 109. The method of any listed embodiment, such as number 106, wherein protecting comprises reducing the activity of the chemical stress by 80%, 110. The method of any listed embodiment, such as number 94, wherein protecting comprises reducing the effective concentration of the chemical stress by 30%, 111. The method of any listed embodiment, such as number 94, wherein protecting comprises reducing the effective concentration of the chemical stress by 50%, 112. The method of any listed embodiment, such as number 94, wherein protecting comprises reducing the effective concentration of the chemical stress by 800%, 113 The method of any listed embodiment, such as number 94, wherein protecting comprises reducing the effective concentration of the chemical stress by 90%, 114. The method of any listed embodiment, such as number 94, wherein protecting comprises reducing the effective concentration of the chemical stress by 95%. 115. The method of any listed embodiment, such as number 76, wherein preventing the chemical stress from being active in the interior of the droplet comprises excluding the chemical stress moiety from the interior of the droplet. 116. The method of any listed embodiment, such as number 115, wherein excluding the chemical stress moiety from the interior of the droplet comprises flushing the droplet using a gas. 117. The method of any listed embodiment, such as number 116, wherein the gas comprises Argon. 118. The method of any listed embodiment, such as number 116, wherein the gas is Argon gas. 119. The method of any listed embodiment, such as number 116, wherein the gas comprises a noble gas. 120. The method of any listed embodiment, such as number 116, wherein the gas comprises nitrogen gas. 121. The method of any listed embodiment, such as number 116, wherein the gas is nitrogen gas. 122. The method of any listed embodiment, such as number 116, wherein the gas excludes oxygen from the droplet. 123. The method of any listed embodiment, such as number 115, wherein excluding the chemical stress moiety comprises reducing the activity of the chemical stress by at least 30%. 124. The method of any listed embodiment, such as number 115, wherein excluding the chemical stress moiety comprises reducing the activity of the chemical stress by at least 50%. 125. The method of any listed embodiment, such as number 115, wherein excluding the chemical stress moiety comprises reducing the activity of the chemical stress by at least 80%. 126 The method of any listed embodiment, such as number 115, wherein excluding the chemical stress moiety comprises reducing the activity of the chemical stress by at least 90% 127. The method of any listed embodiment, such as number 115, wherein excluding the chemical stress moiety comprises reducing the activity of the chemical stress by at least 95%. 128. The method of any listed embodiment, such as number 115, wherein excluding the chemical stress moiety comprises reducing the effective concentration of the chemical stress by at least 30%. 129. The method of any listed embodiment, such as number 115, wherein excluding the chemical stress moiety comprises reducing the effective concentration of the chemical stress by at least 50%. 130. The method of any listed embodiment, such as number 115, wherein excluding the chemical stress moiety comprises reducing the effective concentration of the chemical stress by at least 80%. 131. The method of any listed embodiment, such as number 115, wherein excluding the chemical stress moiety comprises reducing the effective concentration of the chemical stress by at least 90%. 132. The method of any listed embodiment, such as number 115, wherein excluding the chemical stress moiety comprises reducing the effective concentration of the chemical stress by at least 95%. 133. The method of any listed embodiment, such as number 76, wherein allowing the chemical stress moiety to mediate crosslinking at the droplet perimeter comprises providing the chemical stress moiety at the droplet perimeter. 134. The method of any listed embodiment, such as number 76, wherein allowing the chemical stress moiety to mediate crosslinking at the droplet perimeter comprises providing the chemical stress moiety in an immiscible carrier harboring the droplet. 135. The method of any listed embodiment, such as number 76, wherein allowing the chemical stress moiety to mediate crosslinking at the droplet perimeter comprises inducing chemical stress in an immiscible carrier harboring the droplet. 136. The method of any listed embodiment, such as number 135, wherein the chemical stress moiety is selected from the list consisting of an oxidative stress moiety, a free radical moiety, a phosphoryl radical, benzoyl radical, an LAP [Lithium phenyl (2,4,6-trimethylbenzoyl) phosphinate cleavage radical, a methyl radical, a hydroxyl radical (OH·), superoxide anion (·O2-), hydrogen peroxide (H2O2), singlet oxygen (1O2), and ozone (O3). 137. The method of any listed embodiment, such as number 135, wherein the chemical stress moiety is LAP. 138. The method of any listed embodiment, such as number 135, wherein the inducing comprises subjecting the immiscible carrier to electromagnetic radiation. 139 The method of any listed embodiment, such as number 138, wherein the electromagnetic radiation comprises visible, near ultraviolet or ultraviolet radiation 140. The method of any listed embodiment, such as number 138, wherein the electromagnetic radiation induces oxidative stress. 141. The method of any listed embodiment, such as number 76, wherein the ameliorating comprises encapsulating active biomolecules such that at least 75% of encapsulated biomolecules retain an activity exhibited prior to encapsulating. 142. The method of any listed embodiment, such as number 141, wherein at least 90% of encapsulated biomolecules retain an activity exhibited prior to encapsulating. 143. The method of any listed embodiment, such as number 76, wherein the ameliorating comprises encapsulating active biomolecules such that effective concentration of the biomolecules in a resulting microcapsule population is at least 75% of an effective concentration of the encapsulated biomolecules prior to encapsulating. 144. The method of any listed embodiment, such as number 143, wherein the concentration is at least 90% of an effective concentration of the encapsulated biomolecules prior to encapsulating. 145. The method of any listed embodiment, such as number 76, wherein the ameliorating comprises encapsulating active biomolecules such that effective biomolecule activity in a resulting microcapsule population is at least 75% of an effective biomolecule activity of the encapsulated biomolecules prior to encapsulating. 146. The method of any listed embodiment, such as number 145, wherein the effective biomolecule activity in a resulting microcapsule population is at least 90% of an effective biomolecule activity of the encapsulated biomolecules prior to encapsulating. 147. The method of any listed embodiment, such as number 76, wherein the ameliorating comprises encapsulating live cells such that cell viability in a resulting microcapsule population is at least 75% of cell viability prior to encapsulating. 148. The method of any listed embodiment, such as number 147, wherein the cell viability in a resulting microcapsule population is at least 90% of cell viability prior to encapsulating. 149. The method of any listed embodiment, such as number 147, wherein the cell viability in a resulting microcapsule population is at least 70% of cell viability prior to encapsulating. 150. The method of any listed embodiment, such as number 147, wherein the cell viability in a resulting microcapsule population is at least 50% of cell viability prior to encapsulating. 151. The method of any listed embodiment, such as number 147, wherein the cell viability in a resulting microcapsule population is at least 30% of cell viability prior to encapsulating 152. A composition comprising a population of microcapsules, wherein at least some of the microcapsules harbor living cells such that a first microcapsule comprises a liquid core harboring a first cell and a second microcapsule comprises a liquid core harboring a second cell, and wherein the frequency of living cells in the population of microcapsules is no less than 30% of the total number of cells in the population of microcapsules. 153. The composition of any listed embodiment, such as number 152, wherein the first microcapsule comprises a crosslinked hydrogel shell. 154. The composition of any listed embodiment, such as number 152, wherein the frequency is at least 80%, 155. The composition of any listed embodiment, such as number 152, wherein the frequency is at least 90%, 156. The composition of any listed embodiment, such as number 152, wherein the frequency is at least 95%. 157. The composition of any listed embodiment, such as number 152, wherein at least some of the living cells proliferate to form colonies within microcapsules of the population. 158. The composition of any listed embodiment, such as number 157, wherein at least 75% of the viable cells proliferate to form colonies within microcapsules of the population. 159. The composition of any listed embodiment, such as number 157, wherein at least 85% of the viable cells proliferate to form colonies within microcapsules of the population. 160. The composition of any listed embodiment, such as number 152, wherein forming the composition comprises excluding an oxidative stress from a precursor to the liquid core of the first microcapsule. 161. The composition of any listed embodiment, such as number 152, wherein forming the composition does not comprise inducing an oxidative stress to crosslinking a hydrogel shell. 162. The composition of any listed embodiment, such as number 152, wherein the microcapsules are porous to biomolecules of a size equivalent to less than 500 bases. 163. A composition comprising an emulsion droplet in an immiscible carrier, wherein the droplet comprises a biomolecule, wherein the immiscible carrier comprises an oxidative stress inducer activated to generate an oxidative stress, and wherein the oxidative stress does not inactivate the biomolecule. 164. The composition of any listed embodiment, such as number 163, wherein the droplet comprises a redox modulator. 165. The composition of any listed embodiment, such as number 163, wherein the droplet comprises DTT. 166. The composition of any listed embodiment, such as number 163, wherein the droplet comprises an oxidative stress molecule exclusion agent. 167. The composition of any listed embodiment, such as number 163, wherein the droplet comprises a noble gas. 168. The composition of any listed embodiment, such as number 163, wherein the droplet comprises Argon. 169. The composition of any listed embodiment, such as number 163, wherein the droplet comprises Nitrogen gas. 170. The composition of any listed embodiment, such as number 163, wherein the biomolecule comprises a nucleic acid 171. The composition of any listed embodiment, such as number 163, wherein the biomolecule comprises a viable cell. 172. The composition of any listed embodiment, such as number 163, wherein the oxidative stress inducer is selected from the list consisting of an oxidative stress moiety, a free radical moiety, a phosphoryl radical, benzoyl radical, an LAP [Lithium phenyl (2,4,6-trimethylbenzoyl) phosphinate cleavage radical, a methyl radical, a hydroxyl radical (OH·), superoxide anion (·O2-), hydrogen peroxide (H2O2), singlet oxygen (1O2), and ozone (O3). 173. The composition of any listed embodiment, such as number 163, wherein the oxidative stress inducer comprises LAP. 174. The composition of any listed embodiment, such as number 163, wherein the oxidative stress inducer is activated by UV irradiation. 175. The composition of any listed embodiment, such as number 163, wherein the oxidative stress inducer is activated by visible light irradiation. 176 The composition of any listed embodiment, such as number 163, wherein the oxidative stress is quenched in the interior of the droplet. 177. The composition of any listed embodiment, such as number 163, wherein the oxidative stress is excluded from the interior of the droplet. 178. The composition of any listed embodiment, such as number 171, wherein the cell faces a risk of oxidative stress mediated mortality of no greater than 25%. 179. The composition of any listed embodiment, such as number 178, wherein the risk is measured by assaying a population of microcapsules arising from an emulsion of which the emulsion droplet is a constituent. 180. The composition of any listed embodiment, such as number 171, wherein the cell faces a risk of oxidative stress mediated mortality of no greater than 10%. 181. The composition of any listed embodiment, such as number 171, wherein the cell faces a risk of oxidative stress mediated mortality of no greater than 5%.

### EXAMPLES

Example 1. Combination microcapsule preparation for Eukaryotic cell microcapsules. This example demonstrates the use of a combination approach to encapsulate eukaryotic K-562 cells within microcapsules.

Cell Preparation. K-562 cells were cultivated in RPMI-1640 supplemented with 10% FBS and 1× Penicillin-Streptomycin at 37° C. in the presence of 5% CO₂. Before the experiment, 0.5 mL of cell culture was centrifuged at 300 g for 5 min. and resuspended in 0.5 ml of 1×PBS Cell concentration was evaluated by counting them using a hemocytometer. Cell concentration was adjusted to 2 min/mL in 1×PBS.

Encapsulation. Shell solution was prepared by mixing 50 μL 2× shell polymer (Droplet Genomics, Shell 1090) with 50 μL of 100 mM BICINE buffer (pH 8.6). Core solution was prepared by mixing 50 μL core polymer (Droplet Genomics, 20% Dextran 500) with 25 μL of 2 mln/mL cell suspension in 1×PBS, 24 μL 100 mM BICINE buffer (pH 8.6), and 1 μL 1M DTT (Sigma-Aldrich, 43816). 100 μL of the working solutions were added into two different 1 mL syringes back-filled with ~300 μL HFE-7500 (Sigma-Aldrich, 98-0212-2929-3) and 1 mL of 0.25% DSO (Droplet Genomics, DG-DSO-20) was added into another 1 mL syringe. SPCs were generated with flow rates of 150 μl/hr; 150 μl/hr; 600 μL/hr for shell, core, and DSO, respectively, using the inDrop microfluidic device (Droplet Genomics). The emulsion was collected into a 1.5 mL tube with 200 μL of light mineral oil (Sigma, 330779-1L).

Polymerization. The shell gelation was performed by transferring the collected emulsion at 37° C. for 30 min. After the DTT-induced gelation, excess oil was removed. SPCs were recovered using 100 μL of 20% PFO (Fluorochem, 007128), 500 μL ice-cold capsule recovery buffer, and 500 μL ice-cold RPMI-1640 medium. 1 mL of SPCs was transferred into a new 1.5 mL tube and supplemented with 0.1% LAP, followed by photopolymerization at 405 nm for 20 seconds. Immediately after the photopolymerization, 400-500 μL of ice-cold RPMI-1640 medium was added to the capsule suspension. Capsules were washed twice with ice-cold RPMI-1640 medium. During the washing steps, centrifugations were performed at 300 g for 1 min., at +4° C.

Microcapsule cell culturing. After the washing steps, closely packed capsules were transferred into a 6 cm Petri dish prefilled with 5 mL of warm RPMI-1640 medium. Spheroid cultivation was carried out at 37° C. in the presence of 5% CO2.

The results are shown in FIG. 2.

This Example demonstrates that the combination of DTT-induced gelation and photopolymerization allows the generation of stable, well-centered >150 μm SPCs.

Example 2. Microcapsule population comprising proliferating eukaryotic cells. A microcapsule population is generated according to the protocol of Example 1. The microcapsules are incubated under growth conditions and are assayed for colony formation after six days in culture.

An image of the resulting colonies is shown in FIG. 3.

About half of the microcapsules (14/28) are observed to contain K 562 cells. Of the microcapsules containing cells, about 3/14 are observed to have supported cell proliferation. This indicates that although K 562 cells are very vulnerable to the encapsulation process, the method of Example 1 was able to successfully encapsulate viable cells capable of proliferation.

Example 3. Crosslinking using 1:90 DexMbAB linked by DTT is compatible with K562 cell encapsulation.

Different encapsulation conditions were evaluated as to their microcapsule formation and compatibility with the K562 cell-line. The following reagents were used for the assessment.
Shell: 125 uL 2× shell, lot: 20221228GZ; 125 uL pH 8.6 BICINE 0.1 M
Core: 125 uL 2× core, lot: 20221018GZ; 2.5 uL 1M DTT; 31 uL K562+RPMI complete media; 91.5 uL pH 8.6 BICINE 0.1M. Oil: 0.25% DSO

K562 cells were resuspended in complete RPMI media, measured concentration: 8.12×10\hat{ }6 cells/ml; 93% live. Emulsions were generated at Flow rates: 75/75/450 uL/h for core/shell/oil. The predicted occupancy should be ~7-8%.

The above reagents were subjected to three distinct Sample Conditions. Sample Condition 1 omitted emulsion incubation between collection and emulsion breaking. This incubation is necessary to allow shell cross-linking by DTT to occur. In addition, this condition included exposure to 405 nm light to demonstrate that it is not the step causing shell formation when the photo-initiator is excluded. The Condition steps comprised exposing the emulsion to 405 nm for 30 s, breaking the emulsion with 1×PBS+20% PFO, washing 3× with 1×PBS+0.1 Pluronic, and bringing SPCs to 25 cm\hat{ }2 flask and adding 5 mL complete RPMI media.

Sample Condition 2 comprised incubation at room temperature for 45 minutes, followed by breaking the emulsion with 1×PBS+20% PFO, washing 3× with 1×PBS+0.1 Pluronic, and bringing SPCs to 25 cm\hat{ }2 flask and adding 5 mL complete RPMI media.

Sample Condition 3 comprised incubation at 4 C for 45 minutes, followed by breaking the emulsion with 1×PBS+20% PFO, washing 3× with 1×PBS+0.1 Pluronic, and bringing SPCs to 25 cm\hat{ }2 flask and adding 5 mL complete RPMI media.

Microcapsule populations generated through each of the three sample conditions were incubated at 37 deg, with 5% CO2 for six days, scored and imaged.

Few microcapsules generated through Sample Condition 1 were evident by the end of the six day incubation period. The sample comprised an abundance of broken shells and free cells, but only a few microcapsules were evident, and these showed very weak microcapsule shells.

An image of these results is shown in FIG. 4. Two microcapsules are weakly evident below the asterisk.

Microcapsules harboring proliferating K562 cells were evident on the assays of Sample Condition 2, shown in FIG. 5, and Sample Condition 3, shown in FIG. 6. Both sample conditions yielded regular, 80 um microcapsules, a proportion of which harbored proliferating cells. The proportion of microcapsules harboring proliferating K562 cells was similar to the proportion expected (7%-8%) given the starting reagent concentrations.

This set of experiments demonstrated that forming microcapsules without the photo-inducer LAP, but with DTT, yields microcapsules that can be manipulated in aqueous phase. It also shows that sufficient time needs to be allowed for DTT-based microcapsule formation, referred to as “gelation”, to occur. At the DTT concentration tested, 45 min at room temperature or 4° C. was shown to be long enough for gelation to occur and yield stable microcapsule populations with a relatively high K562 survival rate. In addition, we confirmed that exposure to 405 nm light is not inducing to polymerization in the absence of a photo-initiator.

Example 4. Polymerization using droplet-excluded photo-initiators. Carrier-soluble photo-initiators were used to induce free-radical formation in the vicinity of the edges of emulsion droplets upon treatment with high energy light. A broad range of photo-inducers were used, all of which were successful in inducing microcapsule formation. This example indicates that exclusion of photo-inducers from the emulsion droplet interior is a viable approach to microcapsule formation. This approach does not uniformly generate free radicals within the interior of the microcapsule precursor droplets. Rather, free radicals or chemical stress molecules contact the microcapsule precursors at the microcapsule perimeter.

Details of the experiments performed are as follows.

Aqueous emulsions were formed, excess HFE removed, and photo-initiator dissolved in HFE7500 added. Emulsions were vortexed and exposed to 405 nm LED light for 30 seconds. Emulsions were imaged after polymerization in oil and after breaking in aqueous phase.

Emulsions were generated as follows. Core: mix 100 uL of 20% m/m Dextran 500, with 100 uL×1 PBS, load to the syringe. Shell: mix 100 uL of 20% in/nm DexMAB1090, with 20 uL 100 mM DTT in water, with 80 uL nuclease free water. For oil phase, use 0.25% of surfactant. Chip: CED-40. Speed: 75/75/450 uL/h for core/shell/oil respectively.

The following reagents were used.

TABLE 1

| Reagent | | | Catalogue | Lot number |
|---|---|---|---|---|
| category | Name of reagent | Manufacturer | number | (if applicable) |
| Microfluidics | DSO in HFE7500 | DG | N/A | 0.25%, 322313HS, |
| | | | | ae20211001 |
| | HFE7500 | 3M | 98021229285 | 21562 |
| | 20% m/v, dextran 500 | Sigma Aldrich | 31392 | BCCF8905 |
| | 20% m/v dextran | DG | DexMAB1090 | GZ20220124GZ25 |
| | MAB1090 | | | |
| | 10x PBS | Invitrogen ™/ | AM9625 | 01025912 |
| | | Thermo Fisher | | |
| | | Scientific | | |
| | DTT, 100 mM | DG | | 20211110GZ |
| | Chip | Droplet Genomics | CED-40 | N/A |
| | Syringe, 1 mL | BBraun Injekt-F | 9166017V | N/A |
| | Needle | Terumo Agani | AN*2316R1 | N/A |
| | PTFE tubing | Adtech | TX/FLUOR 0.56 | N/A |
| | | | mm × 1.07 mm | |
| | 1.5 mL tubes | N/A | N/A | N/A |
| Breaking and | PFO in HFE7500 | Fluorochem | 007128 | 20% v/v, lot |
| lysis reagents | | | | #FCC 553746 |
| | Nuclease free water | Invitrogen ™/ | AM9932 | N/A |
| | | Thermo Fisher | | |
| | | Scientific | | |
| | 1M Tris-HCl, pH 8.0 | Invitrogen ™/ | 15568025 | N/A |
| | | Thermo Fisher | | |
| | | Scientific | | |
| | Triton X-100 | Carl Roth | 3051.3 | 442192447 |
| | KOII, 50% | Carl Roth | 7949.1 | 322186264 |
| | 0.5M EDTA, pH 8.0 | Invitrogen ™/ | 15575-038 | 2120928 |
| | | Thermo Fisher | | |
| | | Scientific | | |
| | DL-Dithiothreitol solution, | Sigma Aldrich | 43816-10 mL | N/A |
| | DTT, 1M in water | | | |
| Initiators | 2-Ethylhexy1 4-(dimethyl- | TCI Chemicals | D1871 | SJ6OC-EI |
| | amino)benzoate, EHDAB | | | |
| | Tetramethylethylene- | From Lab | N/A | N/A |
| | diamine, TEMED | | | |
| | Diphenyl(2,4,6- | TCI Chemicals | D3358 | 7US3K-AP |
| | trimethylbenzoyl)phosphine | | | |
| | oxide, TPO | | | |
| | Lithium phenyl-2,4,6- | TCI Chemicals | L0290 | 2TNLO-SK |
| | trimethylbenzoyl- | | | |
| | phosphinate, LAP | | | |
| | Phenylbis(2,4,6- | TCI Chemicals | P2312 | OU5OA-GC |
| | trimethylbenzoyl)phosphine | | | |
| | oxide, BAPO | | | |
| | 9,10-Phenanthrenequinone, | TCI Chemicals | P0080 | VMQFF-HM |
| | PheQui | | | |
| | 4,4′- | TCI Chemicals | B0139 | T285B-DN |
| | Bis(diethylamino)benzo- | | | |
| | phenone, BABPhe | | | |
| | Methyl benzoylformate, | TCI Chemicals | B1033 | CHWGA-CD |
| | MeBForm | | | |
| | Thioxanthen-9-one, | TCI Chemicals | T2351 | MR6ME-SN |
| | ThioXan | | | |
| | 0.2 um syringe filter | Cytiva Whatman ™ | 15206869 | N/A |
| | 33 mm Ø, PES | Puradisc ™/Fisher | | |
| | | Scientific | | |

The following carrier soluble or carrier dispersed photo-initiators were used: Diphenyl(2,4,6-(timethylbenzoyl)phosphine oxide, TPO; Lithium phenyl-2,4,6-trimethylbenzoylphosphinate, LAP; Phenylbis(2,4,6-trimethylbenzoyl)phosphine oxide, BAPO; 9,10-Phenanthrenequinone, PheQui; 4,4′-Bis(diethylamino)benzophenone, BABPhe; Methyl benzoylformate, MeBForm: Thioxanthen-9-one, ThioXan. Tetramethylethylenediamine, TEMED or 2-Ethylhexyl 4-(dimethylamino)benzoate, EHDAB was used as an additive. Initiators and additives were used at a final concentration of 0.25M in the carrier.

After exposure to light, emulsions were broken using 20% PFO v/v in HFE7500 and Tris-HCl+0.1% X−100.

The following results were observed.

Microcapsules generated through the combinations above were imaged and presented herein. FIG. 7 shows microcapsules generated through combination “3w” in oil phase prior to breaking their emulsion. FIG. 8 shows microcapsules generated through combination “3o” in aqueous phase. FIG. 9 shows microcapsules generated through combination “3w” in aqueous phase.

Polymerization worked in all cases. Both dispersions and saturated solutions formed microcapsules. Saturated solutions were less likely to yield crosslinked microcapsules. Polymerization yields consistent uniform microcapsule populations, with uniform, symmetrical shells, of approximately the same thickness as if initiator were used in shell phase.

Sample further treated with freshly prepared alkaline lysis reagent mixture comprising 0.5M KOH for 15 min, rt and emulsion stability evaluated by eye. Lysis mix (30-40 uL), incubate for 15 min at room temperature. Each microcapsule population readily dissolved upon alkaline treatment, indicating that microcapsules were fragile but that microcapsule contents could be readily released through chemical manipulation.

Example 5. Argon flushed microcapsule formation supports K562 cells. The effect of flushing microcapsule reagents with Argon was assessed, in combination with free radical protectants DTT and ascorbic acid (AA), on a standard DexMAB polymerization composition to which K562 cells were added.

K562 cells were grown in RPM11640+FBS+P/S, and passaged two days before introduction into the microcapsules Cells were pelleted at 300 g for 5 mi in two 1.5-mL tubes, resuspended in 1×PBS, mixed and re-pelleted. Supernatant was discarded and 2× core solution was added, to 100 uL. Aim for 400-500 k cells. Optionally, 4 uL of freshly prepared ascorbic acid (AA) solution in water (60 mg/mL) was added to final approximately 6 mM concentration. The suspension was diluted to a final volume of 200 uL with 1×PBS and loaded into a syringe.

Shell solution was prepared as follows. 100 uL 2× shell reagent (20% DexMAB1090), 50 uL. 2× shell additive (0.4% LAP), and optionally 20 μL of freshly prepared DTT in water (Prepared 80 mg/mL solution, diluted 1:9) solution were added to a final approximate 6 mM concentration, and the composition was diluted to a final volume of 200 uL with 1×PBS.

Emulsions were generated using a flow rate of 65-80/65-80/400-500. Emulsions were collected in 2-ml tubes without LMO.

Emulsions were exposed to 405 nm light for 20 s. Ar-flushed emulsions were flushed with Argon for 2 min prior to and continuing through a 20 s exposure to 405 nm light. Crosslinked microcapsules were allowed to sit 3-5 min. 300 μL of 1×PBS with 0.1% Pluronic F68+300 μL of 20% PFO, was added and the composition mixed with pipette.

The bottom oil phase was removed, the compositions were washed twice in 1×PBS with 0.1% Pluronic F68, 300 g 5 min spins and then 100 μL of washed emulsion were transferred to 5 ml of complete media (rpmi1640+glutamax+p/s+fbs) in 25-cm2 flasks so as to replace the microcapsule contents with grow medium. Microcapsules and contained cells were cultured at 37° C. for a few days.

It was observed that Standard and Ar-flushed microcapsules formed and were able to support K562 cell growth when the shell composition was supplemented with 6 mM DTT. In the absence of DTT, Ar-flushed microcapsules formed and were able to support K562 cell growth, while standard microcapsules were unstable Addition of 6 mM AA to the aqueous core precursor resulted in microcapsule formation but no K562 growth, independent of Ar flushing.

This example indicates that AR flushing does not interfere with microcapsule formation and supports eukaryotic and fragile cell growth.

Example 6. Microcapsule populations representative of source sample cell diversity. A dissociated tumor cell population is generated. The cell population comprises durable, semi-quiescent core cells and peripheral tumor stem cells at a ratio of about 9:1. The quiescent cells are tolerant of chemical stress, while the peripheral stem cells are very stress sensitive. A subset of the stem cells, but not the quiescent core cells, harbor a mutation associated with tumor metastasis that is readily targeted by an antioncogenic drug.

The first microcapsule population is made from an encapsulation approach in the art. The approach subjects sample cells to chemical stress that selectively kills the stem cells. The resulting microcapsule population comprises quiescent core tumor cells that do not harbor the mutation associated with tumor metastasis.

The microcapsules are subjected to reagent replacement to identify growth medium, and the cells are induced to proliferate. Cells are processed to generate sequence libraries within the microcapsules, and the libraries are released and subjected to sequencing. The mutation associated with tumor metastasis is not identified.

A second microcapsule population is made from an encapsulation approach as disclosed herein. The approach protects sample cells from chemical stress, such that the distribution of core cells and stem cells in the microcapsule population reflects the distribution of core and stem cells in the original tumor. The resulting microcapsule population comprises quiescent core tumor cells at a ratio of 9:1 relative to stem cells.

The microcapsules are subjected to reagent replacement to identify growth medium, and both the core quiescent cells and the stem cells are induced to proliferate. Cells are processed to generate sequence libraries within the microcapsules, and the libraries are released by application of dextranase to the microcapsules and subjected to sequencing. Both types of cells are analyzed, the mutation associated with tumor metastasis is identified, and a suitable pharmaceutical treatment is identified.

This example illustrates a benefit of using a gentle microcapsule generation process that preserves the proportional diversity of the starting sample rather than selecting for a stress-resistant subset of a sample. This example also illustrates a benefit of reagent exchange in that cells may be induced to grow in situ, and then may be processed to from sequencing libraries that are released under physiological conditions.

Example 7. Environmental sample analysis and growth condition assessment. A novel biochemical reaction is observed at an environmental site. The reaction is believed to be driven by an organism in the sample, but the organism is not successfully isolated or cultured.

An environmental sample is obtained and subdivided into microcapsules using an approach herein A subset of the microcapsule population is incubated in standard growth media and some microcapsules are observed to harbor viable, proliferating cell populations, but none are correlated with the novel biochemical reaction.

A second subset of the microcapsules are cultured in a slurry generated from soil obtained from the source of the environmental sample. Some microcapsules are observed to harbor viable, proliferating cell populations. Organisms in some microcapsules are observed to perform the biochemical reaction of interest in a functional assay that involves a measurement of product concentration. These populations are isolated, and their nucleic acids are released under conditions that do not harm the nucleic acids, and they are subjected to sequencing. It is determined that one population of interest has a genome that encodes enzymes consistent with the novel biochemical reaction.

Subsequent subsets of the microcapsules are cultured in subfractions of the soil slurry. Soil subfractions are generated that support growth of cells of the population of interest in microcapsules.

An artificial medium comprising components of the soil subfractions is produced, and it is shown to support growth of cells of the population of interest.

Example 8. The effects of addition of an oxidative stress reducer on a biochemical reaction was assayed through a PCR assay on encapsulated isolated nucleic acid fragments pBR322 plasmids were isolated and encapsulated in microcapsules. The microcapsules were subjected to various UV treatment in the presence and absence of the oxidative stress quencher DTT, and PCR yield was measured as represented by nucleic acid content of the microcapsules subsequent to thermocycling.

Microcapsules were prepared as follows: SPC stock were washed three times with 1 ml 1×PBS solution. Aliquots of SPCs were contacted either with a mixture of 138.5 μl of 1×PBS, 1 μl DTT, and 12.5 μl of 4% LAP, or 1×PBS, 4% LAP in the absence of DTT and allowed to equilibrate. The SPCs were then subjected to either 0, 30 seconds or 90 seconds of JV exposure, simulating the oxidative stress generated when UV is applied to LAP formulations pursuant to microcapsule formation.

Microcapsules were washed to clear LAP solution and then incubated in PCR amplification solution comprising primers selected to generate a 500 bp amplicon and Phire Tissue Direct PCR Master Mix. Microcapsules were then subjected to 35 cycles of denaturation for 20 s at 98 C, annealing for 5 s at 65 Cand extension for 40 s at 72 C, followed by a final extension at 72 C for 1 minute.

Microcapsules were washed and resuspended in wash buffer and SYTO-9 nucleic acid stain to 1 uM. SPCs were then assayed for fluorescence indicative of PCR amplicon formation, and DNA occupancy rates were calculated. Occupancy rates for SPC populations treated with UV LAP in the presence and absence of DTT were as follows.

TABLE 3
Amplicon occupancy

| | 0 s UV/ | 30 s UV/ | 90 s UV/ |
|---|---|---|---|
| | Amplicon | Amplicon | Amplicon |
| Sample: pBR322 | Occupancy | Occupancy | Occupancy |
| LAP + DTT | 0.37 | 0.36 | 0.36 |
| LAP | 0.42 | 0.028 | 0.017 |

The results indicate that IV triggered LAP induction of hydrogel formation had a substantial negative effect on biomolecules exposed thereto, as indicated by the over 10× decrease in amplicon occupancy upon UV treatment of microcapsules harboring LAP in proximity to pBR322 template. That is, pBR322 suitability as a PCR amplification template was negatively impacted by UV/LAP generated oxidative stress such as that generated pursuant to SPC hydrogel formation.

Addition of the oxidative stress protectant DTT completely counteracted this negative effect on the assayed biomolecules. That is, in the presence of DTT added to the SPC interior, in proximity to the pBR322 template, UV/LAP treatment had no deleterious effect on amplicon occupancy, as evidenced by the effectively steady 0.37 to 0.36 occupancy rate independent of IV exposure time. Presence of an oxidative stress scavenging compound, so as to prevent the oxidative stress from contacting the pBR322 templates, protected the microcapsule biocontents from the negative impact of the U V/LAP induced oxidative stress.

This example demonstrates the positive effect on biomolecules of preventing oxidative stress moieties from contacting the biomolecules.

Example 9. Argon flushing increases viability of encapsulated cells. This example demonstrates that excluding oxidative stress conducting moieties from emulsion droplets protects the droplet interior from oxidative stress related damage that may otherwise occur pursuant to hydrogel formation.

K562 cells were grown in RPMI1640+FBS+P/S, p10, and passaged one day prior to emulsification. Compositions were configured to have 0.1% LAP final in DexMAB1090.

SPC emulsions were generated and separated prior to polymerization. A control first portion was subjected to a 20 second polymerizing exposure at 405 nm, while a second portion was washed with Argon gas for 2 minutes prior and continuing through a 20 second polymerizing exposure at 405 nm. To flush with Argon, a piece of microfluidic tubing was attached to a source of argon, the open end of the tubing was placed at the bottom of the tube with the emulsion, and Argon was gently bubbled through the emulsion to avoid splashing.

Microcapsules were maintained in cell growth conditions for several days, and cell growth in control microcapsules was compared to that of Ar-flushed microcapsules.

A full protocol is as follows.

Prep cells: 1. Pellet at 300 g for 5 min in two 2-ml tubes, discard supernatant 2. Resuspend in 300 ul of 1×PBS, collect cells from both tubes 3. Count (result: 2.9M/ml). 4. Mix 250 ul of cells+250 ul of 2× core reagent, use pipette tips for cells 5. Check viability with trypan blue. Result: 99%. 6. Load into syringe.

Prep shell 7. 125 ul of 2×LAP (0.4%)+125 ul of 1×PBS+250 ul of 2× shell reagent (dexmab 10:90) 8. Load into syringe

Encapsulation 9. 40×40 (U91.2) chip 10. Flow rates 100/100/500. 11. Collect in 2-ml tube without LMO.

Light-induced polymerization, breaking droplets, seeding 12. Expose to 405 nm light in for 20 s: a. Control: close tube and expose 20 s b. Ar-flushed flush with Argon for 2 min followed by 20 s exposure to 405 nm light (while continuing flushing with Argon) 13. Let sit 3-5 min. 14. Add 300 ul of 1×PBS with 0.1% Pluronic F68+300 ul of 20% PFO. 15. Remove the bottom oil phase 16. Wash 2× in 1×PBS with 0.1% Pluronic F68, 300 g 5 min spins 17. Transfer each conditions (control and Ar-flushed) to 5 ml of complete media (rpmi1640+glutamax+p/s+fbs) in 25-cm2 flasks. 18. Culture at 37° C. for a few days. 19. Take pictures at timepoint 0, 2, 3, 7 days. 20. Change media (pellet SPCs at 300 g for 5 mm) on day 3.

Argon flushing was associated with some SPC clumping but did not affect cell viability. As seen in FIG. 10, Argon-flushed SPCs exhibited substantially more cell proliferation than did control SPCs. This is particularly evident at seven days after SPC formation, as the cells have had the longest time to proliferate.

Cell proliferation was also evidenced by yellow discoloration of media in which Argon treated SPCs were cultured, indicative of media reagent consumption, relative to the clear color of media in which control SPCs were incubated.

This example illustrates that exclusion of an oxidative stress conducting moiety from an SPC precursor core protects biomolecules or cells from harm such as oxidative stress associated with hydrogel formation.

Example 10. Photoinduced crosslinking is effected while preserving high cell viability and proliferative potential. This example illustrates how taking precautions to reduce oxidative stress results in high cell survival and high proliferative capacity among surviving cells subjected to microcapsule encapsulation. Rates of survivability for DTT protected cells subjected to oxidative stress microcapsule formation in 10:90 DexMAB were seen to be comparable to those observed for oxidative stress free microcapsulated cells in 60:40 DexMAB, indicating that DTT substantially reduced oxidative stress in the microencapsulation process.

Cells were prepared for encapsulation as follows. Cultivate K562 cells in RPMI medium supplemented with 10% fetal bovine serum and 1× penicillin-streptomycin at 37° C. in the presence of 5% CO2. Change culture medium every 3-4 days. Take 1.5 ml of K562 cell suspension and centrifuge at 300 g for 5 min (room temperature). Remove the supernatant and resuspend cells in 1 ml of complete RPM1 culture medium. Combine 10 ul of cell suspension from step #2 with 10 ul of Trypan Blue dye. Mix gently by pipetting and load 10 ul under the hemocytometer. Count live and dead cells. Take cells from the previous step and centrifuge at 300 g for 5 min (room temperature). Remove the supernatant and resuspended cells in a complete RPMI culture medium. The final concentration of viable cells should be ~7.7M/ml.

Cells were introduced into emulsion droplets and encapsulated in microcapsules as follows. Prepare syringes: load 1 syringe with 1 ml of 0.25% DSO and backfill syringes 2 and 3 with 300 μl of HFE 7500. Prepare the Shell solution: combine 50 μl of 2× Shell polymer (DexMab 10:90) with 50 μl of complete RPMI medium. Mix well by pipetting and transfer the solution to a syringe 2. Prepare the Core solution: combine 50 μl of 2× Core polymer with 1 μl 1M DTT, 12.5 μl 4% LAP solution, and 36.5 μl Cell suspension. Mix well by pipetting and transfer the solution to a syringe 3. Generate droplets for 30 min using CF-60 microfluidic chip and the following flow rates: Shell solution—75 μl/h, Core solution—75 μl/h, DSO—450 μl/h. After encapsulation, remove the bottom oil and continue with the shell photopolymerization under a 405 nm LED device for 30 s. Immediately after the photopolymerization, add 1 ml of room temperature complete culture medium supplemented with 0.2% Shell polymer (DexMab 10:90) and 300 μl of 20% PFO. Gently invert the tube several times. Wait 2-3 minutes until all SPCs are released from the emulsion. Transfer SPCs into a new 1.5 ml tube and spin down at 300 g for 2 mm (room temperature). Remove the supernatant and add 1 ml of complete culture medium (without additives). Transfer SPC suspension into a 6 cm Petri dish prefilled with 4 ml of complete culture medium. Cultivate isolated cells at 37° C. at 5% CO2 Change culture medium every 3-4 days.

In the protocol above, DTT is used in the microcapsule precursor to scavenge oxidative stress moieties generated through the shell photopolymerization performed under a 405 nm LED device for 30 s.

Cells were scored for viability prior to encapsulation, and Microcapsules were scored for viability and proliferative ability of encapsulated cells. Cells were observed to have a viability of 99.7+/−0.6% prior to encapsulation. Cell viability was observed to be 76.7+/−0.3% after encapsulation in 10:90 DexMab. Cell proliferation was observed in 80% of isolated viable cells. Both of these cell viability rates were substantially higher than those observed for comparable cell lines subjected to oxidative stress mediated microcapsule formation in the absence of protectants.

An image of proliferating cells is presented in FIG. 11, which depicts cell proliferation in a microcapsule.

This example indicates that a high degree of cell survival and proliferation among surviving cells can be obtained in encapsulation protocols for which precautions are taken to reduce oxidative stress on cells in the eventual microcapsule core. In this example the precautions included encapsulation in the presence of DTT as an oxidative stress protectant, as well as microcapsule transfer into complete media comprising fetal bovine serum acting as a diluent and potential scavenger of ROS immediately after shell cross-linking.

Example 11. Radical free crosslinking. This example illustrates how encapsulation without inducing oxidative stress results in high cell survival and high proliferative capacity among surviving cells subjected to microcapsule encapsulation.

Cells were prepared for encapsulation as in Example 10, above.

Cell were introduced into emulsion droplets and encapsulated in microcapsules as follows. Prepare the Shell solution: combine 50 μl of 2× Shell polymer having a high methacryloyl and acryloyl modification rate (DexMab 60:40) with 50 μl of complete RPMI medium. Mix well by pipetting and transfer the solution to a syringe 2 Prepare the Core solution: combine 50 μl of 2× Core polymer with 1 μl 1 M DTT, 12.5 μl of complete culture medium, and 36.5 μl of Cell suspension. Mix well by pipetting and transfer the solution to a syringe 3. Generate droplets for 30 min using CF-60 microfluidic chip and the following flow rates: Shell solution—75 μl/h, Core solution—75 μl/h, DSO—450 μl/h. After encapsulation, incubate the emulsion at room temperature for 20 min. After the DTT-induced gelation, remove the bottom oil and add 1 ml of room temperature complete culture medium supplemented with 0.2% Shell polymer (DexMab 10:90) and 300 μl of 20% PFO. Gently invert the tube several times. Wait 2-3 minutes until all SPCs are released from the emulsion. Transfer SPCs into a new 1.5 ml tube and spin down at 300 g for 2 min (room temperature) Remove the supernatant and add 1 ml of complete culture medium (without additives). Transfer SPC suspension into a 6 cm Petri dish prefilled with 4 ml of complete culture medium. Cultivate isolated cells at 37° C. at 5% CO2. Change culture medium every 3-4 days (the detailed description of how to change medium is depicted in FIG. 2).

In the protocol above, DTT is used in the microcapsule precursor to induce hydrogel formation in the absence of an oxidate stress inducer. No UV or other oxidative stress inducer is used to trigger microcapsule formation.

Cells were scored for viability prior to encapsulation, and Microcapsules were scored for viability and proliferative ability of encapsulated cells. Cells were observed to have a viability of 99.7+/−0.6% prior to encapsulation. Cell viability was observed to be 94.1+/−2.6% after encapsulation in 60:40 DexMab. Cell proliferation was observed in 88% of isolated viable cells.

An image of proliferating cells is again presented m FIG. 11, which depicts cell proliferation in a microcapsule.

This example indicates that a high degree of cell survival and proliferation among surviving cells can be obtained in encapsulation protocols that do not involve administering oxidative stress on cells in the eventual microcapsule core. In this example the encapsulation was effected in the presence of DTT but in the absence of an exogenous oxidative stress inducer.

The disclosure is further understood in light of the following claims

We claim:

1.-181. (canceled)

182. A microcapsule population comprising at least 100 microcapsules, each microcapsule comprising a crosslinked shell and an aqueous interior, each aqueous interior comprising a viable cell such that the microcapsule population comprises at least 100 cells.

183. The microcapsule population of claim 182, wherein a cell of the microcapsule population differs in a parameter by no more than 10% from a corresponding cell of a microcapsule precursor sample population.

184. The microcapsule population of claim 183, wherein the parameter comprises transcript accumulation pattern.

185. The microcapsule population of claim 183, wherein the parameter comprises proteome accumulation pattern.

186. The microcapsule population of claim 183, wherein the parameter comprises growth rate.

187. The microcapsule population of claim 183, wherein the parameter comprises cell viability dye fluorescence.

188. The microcapsule population of claim 183, wherein the parameter comprises cell motility.

189. The microcapsule population of claim 182, wherein the at least 100 encapsulated cells differ in proportional cell identity by no more than 10% from a population of cells selected from a microcapsule sample precursor population.

190. The microcapsule population of claim 182, wherein the microcapsule population is degradable under physiological conditions.

191. The microcapsule population of claim 182, wherein the microcapsule population is enzymatically degradable.

192. The microcapsule population of claim 182, wherein the microcapsule population is degradable by treatment with a dextranase.

193. The microcapsule population of claim 182, wherein at least some of the living cells proliferate to form colonies within microcapsules of the population.

194. The microcapsule population of claim 182, wherein at least 75% of the viable cells proliferate to form colonies within microcapsules of the population.

195. The microcapsule population of claim 182, wherein the frequency of living cells in the population of microcapsules is no less than 30% of the total number of cells in the population of microcapsules.

196. The microcapsule population of claim 182, wherein the frequency of living cells in the population of microcapsules is no less than 80% of the total number of cells in the population of microcapsules.

197. The microcapsule population of claim 182, wherein the frequency of living cells in the population of microcapsules is no less than 30% of the total number of microcapsules in the population of microcapsules.

198. The microcapsule population of claim 182, wherein the frequency of living cells in the population of microcapsules is no less than 80% of the total number of microcapsules in the population of microcapsules.

199. The microcapsule population of claim 182, wherein the crosslinked shell comprises an oxidative stress agent absent from the aqueous interior.

200. The microcapsule population of claim 199, wherein dissolved oxygen molecules are displaced from the aqueous interior of the capsule.

201. The microcapsule population of claim 200, wherein the aqueous interior comprises a dissolved gas that excludes oxygen.

202. The microcapsule population of claim 199, wherein the microcapsule population is suspended in an oil carrier comprising a water-insoluble crosslinking initiator.

203. The microcapsule population of claim 202, wherein the water-insoluble crosslinking initiator is present at a concentration insufficient to allow radicals to reach the aqueous interior of the microcapsule.

204. The microcapsule population of claim 182, wherein the crosslinked shell comprises a free radical stress agent absent from the aqueous interior.

205. The microcapsule population of claim 204, wherein the microcapsule population is suspended in an oil carrier comprising a water-insoluble free radical crosslinking initiator.

206. The microcapsule population of claim 205, wherein the water-insoluble crosslinking initiator is present at a concentration insufficient to allow radicals to reach the aqueous interior of the capsule.
